(19) United States
(12) Patent Application Publication
(10) Pub. No.: US 2026/0358273 A1
(43) Pub. Date: Nov. 12, 2026

(54) SYSTEMS AND METHODS FOR AUTONOMOUS MICROCRYSTAL ELECTRON DIFFRACTION MICROSCOPY

(71) Applicant: The Regents of the University of California, Oakland, CA (US)

(72) Inventors: Tamir Gonen, Los Angeles, CA (US); Johan Unge, Los Angeles, CA (US)

(21) Appl. No.: 19/483,044

(22) PCT Filed: May 8, 2024
(86) PCT No.: PCT/US2024/028248

§ 371 Date: Nov. 10, 2025

Related U.S. Application Data
(60) Provisional application No. 63/501,042, filed on May 9, 2023.

(51) Int. Cl. G01N 23/20058

(52) U.S. Cl. CPC G01N 23/20058; G01N 2223/0566

(57) ABSTRACT

Systems and methods for autonomous MicroED microscopy implement and/or include automatically: capturing a low-magnification atlas of a grid including a mixture of compounds; selecting one or more grid squares from the low-magnification atlas; capturing a medium-magnification montage of the grid based on the selected one or more grid squares; setting a eucentric focus of an electron microscope; capturing a continuous rotation MicroED video of at least one crystal from the medium-magnification montage; estimating a crystal physical dimension of the at least one crystal, based on the continuous rotation MicroED video; calculating at least one of a unit cell dimension and a symmetry for the at least one crystal, based on the estimated crystal physical dimension; and outputting a compositional analysis of the mixture of compound based on the at least one of the unit cell dimension and the symmetry.

### CROSS-REFERENCE TO RELATED APPLICATIONS

This application claims priority to and the benefit of U.S. Provisional Application No. 63/501,042, filed May 9, 2023 and titled “Methods for Automation and Compositional Analyses of MicroED Data,” the entire contents of which are herein incorporated by reference for all purposes.

### STATEMENT REGARDING FEDERALLY SPONSORED RESEARCH OR DEVELOPMENT

This invention was made with government support under HDTRA-21-1-0004 awarded by the Defense Threat Reduction Agency, and GM136508 awarded by the National Institutes of Health. The government has certain rights in the invention.

### TECHNICAL FIELD

This disclosure relates generally to chemistry, and, in particular, to systems and methods for determining atomic structures using microcrystal electron diffraction (MicroED).

### BACKGROUND

Various techniques exist for determining the atomic structures of small or large molecules, as long as the single crystals are of good diffraction quality and are substantially larger than 1 μm. However, the available methods are limited when dealing with sub-micron crystals and complex mixtures of compounds or samples with poor diffraction. MicroED is an effective method for analyzing the structural properties of sub-micron crystals, which are frequently found in low-molecule powders.

By developing and using an autonomous and high throughput approach to MicroED, the expansion of capabilities and the possibility of performing complete compositional analysis of complex samples is demonstrated. With the use of SerialEM for data collection of thousands of datasets from thousands of crystals and an automated processing pipeline, compositional analysis of complex mixtures of organic and inorganic compounds can be accurately executed. Reliable compound identification can be accomplished without needing to solve the structures, thereby allowing for a quantitative analysis of samples on the fly even with compounds having very similar chemical properties. These compounds can be distinguished by their crystal structure properties instead of being separated based on chemical properties. Additionally, with sufficient statistics from the autonomous approach, even small amounts of compounds in mixtures can be reliably detected. Finally, atomic structures can be determined from the thousands of data sets.

### SUMMARY

The present disclosure addresses needs in the field by presenting systems and methods for autonomous MicroED, data collection, and processing. Thus, the systems and methods described herein provide for improvements in at least the technical field of analytical chemistry and the characterization of complex mixtures whatever their composition.

According to one aspect of the present disclosure, a MicroED microscopy system is provided. The system comprises an electron microscope configured to automatically receive a grid, the grid including a mixture of compounds; and a controller configured to: cause the electron microscope to capture a low-magnification atlas of the grid, select one or more grid squares from the low-magnification atlas, cause the electron microscope to capture a medium-magnification montage of the grid based on the selected one or more grid squares, set a eucentric focus of the electron microscope, cause the electron microscope to capture a continuous rotation MicroED video of at least one crystal from the medium-magnification montage, estimate a crystal physical dimension of the at least one crystal, based on the continuous rotation MicroED video, calculate at least one of a unit cell dimension and a symmetry for the at least one crystal, based on the estimated crystal physical dimension, and output a compositional analysis of the mixture of compound based on the at least one of the unit cell dimension and the symmetry.

According to another aspect of the present disclosure, a MicroED microscopy method is provided. The method comprises automatically capturing, by an electron microscope, a low-magnification atlas of a grid, the grid including a mixture of compounds; selecting one or more grid squares from the low-magnification atlas; capturing, by the electron microscope, a medium-magnification montage of the grid based on the selected one or more grid squares; setting a eucentric focus of the electron microscope; capturing, by the electron microscope, a continuous rotation MicroED video of at least one crystal from the medium-magnification montage; estimating a crystal physical dimension of the at least one crystal, based on the continuous rotation MicroED video; calculating at least one of a unit cell dimension and a symmetry for the at least one crystal, based on the estimated crystal physical dimension; and outputting a compositional analysis of the mixture of compound based on the at least one of the unit cell dimension and the symmetry.

According to another aspect of the present disclosure, a non-transitory computer-readable medium is provided. The non-transitory computer-readable medium stores instructions that, when executed by at least one processor of a MicroED system, cause the system to automatically perform operations comprising capturing, by an electron microscope, a low-magnification atlas of a grid, the grid including a mixture of compounds; selecting one or more grid squares from the low-magnification atlas; capturing, by the electron microscope, a medium-magnification montage of the grid based on the selected one or more grid squares; setting a eucentric focus of the electron microscope; capturing, by the electron microscope, a continuous rotation MicroED video of at least one crystal from the medium-magnification montage; estimating a crystal physical dimension of the at least one crystal, based on the continuous rotation MicroED video; calculating at least one of a unit cell dimension and a symmetry for the at least one crystal, based on the estimated crystal physical dimension; and outputting a compositional analysis of the mixture of compound based on the at least one of the unit cell dimension and the symmetry.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example workflow for autonomous MicroED data collection and processing, in accordance with various aspects of the present disclosure.

FIG. 2 shows images illustrated crystals that appear to have “melted,” in accordance with various aspects of the present disclosure.

FIG. 3 shows a bar graph illustrating the number of crystals identified for each composition in a mixture, in accordance with various aspects of the present disclosure.

FIG. 4A shows a graph illustrating the number of crystals identified for each composition in a mixture, in accordance with various aspects of the present disclosure.

FIG. 4B shows a graph illustrating the number of crystals identified for each composition in a mixture, in accordance with various aspects of the present disclosure.

FIG. 4C shows a graph illustrating the number of crystals identified for each composition in a mixture, in accordance with various aspects of the present disclosure.

FIG. 4D shows a graph illustrating the number of crystals identified for each composition in a mixture, in accordance with various aspects of the present disclosure.

FIG. 5 shows a series of structures found in analyzed mixtures, in accordance with various aspects of the present disclosure.

FIG. 6 shows a series of a series of graphs illustrating the unit cell parameters from processing, in accordance with various aspects of the present disclosure.

FIG. 7 shows a series of a series of graphs illustrating the unit cell parameters from processing, in accordance with various aspects of the present disclosure.

FIG. 8 shows a series of a series of graphs illustrating the unit cell parameters from processing, in accordance with various aspects of the present disclosure.

FIG. 9 shows a series of a series of graphs illustrating the unit cell parameters from processing, in accordance with various aspects of the present disclosure.

FIG. 10 shows a Pearson correlation plot of unit cell parameters from processing and the reference unit cell parameters, in accordance with various aspects of the present disclosure.

FIG. 11 shows a Pearson correlation plot of unit cell parameters from processing and the reference unit cell parameters, in accordance with various aspects of the present disclosure.

FIG. 12 shows a Pearson correlation plot of unit cell parameters from processing and the reference unit cell parameters, in accordance with various aspects of the present disclosure.

FIG. 13 shows a Pearson correlation plot of unit cell parameters from processing and the reference unit cell parameters, in accordance with various aspects of the present disclosure.

FIG. 14A shows a series of crystal area distribution plots, in accordance with various aspects of the present disclosure.

FIG. 14B shows a series of crystal area distribution plots, in accordance with various aspects of the present disclosure.

FIG. 15 shows a series of crystal structures showing an overlay of the experimental solved structures in a mixture and the literature-reported structures, in accordance with various aspects of the present disclosure.

FIG. 16 shows an example MicroED system in accordance with various aspects of the present disclosure.

FIG. 17 shows an example MicroED method in accordance with various aspects of the present disclosure.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various aspects of the present disclosure. However, it will be apparent to those skilled in the art that the various features, concepts, and aspects described herein may be implemented and practiced without these specific details.

The present disclosure may be implemented on or with the use of computing devices including control units, processors, and/or memory elements in some examples. As used herein, a “control unit” may be any computing device configured to send and/or receive information (e.g., including instructions) to/from various systems and/or devices. A control unit may comprise processing circuitry configured to execute operating routine(s) stored in a memory. The control unit may comprise, for example, a processor, microcontroller, application-specific integrated circuit (ASIC), field-programmable gate array (FPGA), and the like, any other digital and/or analog components, as well as combinations of the foregoing, and may further comprise inputs and outputs for processing control instructions, control signals, drive signals, power signals, sensor signals, and the like. All such computing devices and environments are intended to fall within the meaning of the term “controller,” “control unit,” “processor,” or “processing circuitry” as used herein unless a different meaning is explicitly provided or otherwise clear from the context. The term “control unit” is not limited to a single device with a single processor, but may encompass multiple devices (e.g., computers) linked in a system, devices with multiple processors, special purpose devices, devices with various peripherals and input and output devices, software acting as a computer or server, and combinations of the above. In some implementations, the control unit may be configured to implement cloud processing, for example by invoking a remote processor.

Moreover, as used herein, the term “processor” may include one or more individual electronic processors, each of which may include one or more processing cores, and/or one or more programmable hardware elements. The processor may be or include any type of electronic processing device, including but not limited to central processing units (CPUs), graphics processing units (GPUS), ASICs, FPGAs, microcontrollers, digital signal processors (DSPs), or other devices capable of executing software instructions. When a device is referred to as “including a processor,” one or all of the individual electronic processors may be external to the device (e.g., to implement cloud or distributed computing). In implementations where a device has multiple processors and/or multiple processing cores, individual operations described herein may be performed by any one or more of the microprocessors or processing cores, in series or parallel, in any combination.

As used herein, the term “memory” may be any storage medium, including a non-volatile medium, e.g., a magnetic media or hard disk, optical storage, or flash memory, including read-only memory (ROM), electrically programmable ROM (EPROM), electrically erasable programmable ROM (EEPROM); a volatile medium, such as system memory, e.g., random access memory (RAM) such as dynamic RAM (DRAM), synchronous dynamic RAM (SDRAM), static RAM (SRAM), extended data out (EDO) DRAM, extreme data rate dynamic (XDR) RAM, double data rate (DDR) SDRAM, etc.; on-chip memory; and/or an installation medium where appropriate, such as software media, e.g., a CD-ROM, a DVD-ROM, a Blu-ray disc, or floppy disks, on which programs may be stored and/or data communications may be buffered. The term “memory” may also include other types of memory or combinations thereof. For the avoidance of doubt, cloud storage is contemplated in the definition of memory.

Before any aspects of the invention are explained in detail, it is to be understood that the invention is not limited in its application to the details of construction and the arrangement of components set forth in the following description or illustrated in the following drawings. The invention is capable of other aspects and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

It is also to be understood that any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Thus, a reference to first and second elements does not mean that only two elements may be employed or that the first element must precede the second element in some manner.

Also as used herein, unless otherwise limited or defined, “or” indicates a non-exclusive list of components or operations that can be present in any variety of combinations, rather than an exclusive list of components that can be present only as alternatives to each other. For example, a list of “A, B, or C” indicates options of: A; B; C; A and B; A and C; B and C; and A, B, and C. Correspondingly, the term “or” as used herein is intended to indicate exclusive alternatives only when preceded by terms of exclusivity, such as, e.g., “either,” “one of,” “only one of,” or “exactly one of.” Further, a list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of each of A, B, and C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C. In general, the term “or” as used herein only indicates exclusive alternatives (e.g., “one or the other but not both”) when preceded by terms of exclusivity, such as, e.g., “either,” “one of,” “only one of,” or “exactly one of.”

The present disclosure includes a description of various methods. For any methods described, regardless of whether the method is described in conjunction with a flow diagram, it should be understood that unless otherwise specified or required by context, any explicit or implicit ordering of steps performed in the execution of a method does not necessarily imply that those steps must be performed in the order presented, but instead the steps may be performed in a different order and/or in parallel.

The following discussion is presented to enable a person skilled in the art to make and use aspects of the invention. Various modifications to the illustrated aspects will be readily apparent to those skilled in the art, and the generic principles herein can be applied to other aspects and applications without departing from aspects of the invention. Thus, aspects of the invention are not intended to be limited to aspects shown but are to be accorded the widest scope consistent with the principles and features disclosed herein. The following detailed description is to be read with reference to the figures, in which like elements in different figures have like reference numerals. The figures, which are not necessarily to scale, depict selected aspects and are not intended to limit the scope of aspects of the invention. Skilled artisans will recognize the examples provided herein have many useful alternatives and fall within the scope of aspects of the invention.

Microcrystal electron diffraction (MicroED) is a cryogenic electron microscopy (cryoEM) method for determining the 3D structure of inorganic, organic, and/or biological macromolecules. Compared to X-rays or neutrons, electrons exhibit a stronger interaction with the sample and cause considerably less damage per useful elastic scattering event. Thus, the preferred crystal size for MicroED is well below 1 μm³, and even crystals consisting of only a few layers can be used for structure determination. In fact, electron diffraction is believed to be the only method that can routinely produce a complete diffraction dataset for samples of this size and can be acquired with only picograms of a sample. This is in contrast to X-ray free-electron laser (XFEL) serial crystallography and serial synchrotron crystallography (SSX), which require hundreds of thousands of crystals, with the crystals needing to be larger than 1 μm and 5 μm, respectively. Ultimately, crystallographic techniques that use electrons, X-rays, or neutrons are complementary because the beams interacting with the sample have different sensitivities for various elements. Moreover, due to their relatively larger cross-section as compared with X-rays, MicroED and neutron crystallography can often visualize hydrogen atoms. Finally, electron diffraction can provide information on charge.

Determining the composition of mixtures of compounds is a common and essential task in many fields of chemistry, such as pharmaceuticals, materials science, and environmental chemistry; however, it can be challenging as the components may have similar physical and chemical properties. There are several comparative methods for determining the composition of mixtures, including chromatography, spectroscopy, and mass spectrometry. One comparative method for the analysis of crystalline powder is powder X-ray diffraction (PXRD), which is capable of identifying phases of mixtures with a detection limit of about 5-10% for typical laboratory X-ray diffractometers. This comparative method is, however, limited to identification of compounds with a known crystal structure and diffraction; in fact, the diffraction of all components in the mixture needs to be simulated and compared with the experimental result in order to get unambiguous results. For complex materials, peak overlay may present problems for phase analysis, in particular at high diffraction angles, due to the one-dimensional d-spacings analysis. In order to determine the structure of unknown compounds in such methods, a homogeneous sample is usually needed.

Using PXRD for analytical purposes of samples is complicated by several compounds or phases, diffraction patterns need to be known in advance for all the constituents using the comparative method of X-ray powder diffraction compositional analysis. The content of a mixture with crystalline constituents can be quantitatively analyzed with PXRD using the Reference Intensity Ratio (RIR). Typically it includes preparing a mixture for each of the components with an equal amount of a reference components from which the relative peak heights can be used as a reference. When the samples of unknown ratios are mixed with the reference component the searched for ratios can be estimated. Alternatively, the peak heights can be calculated knowing the constituents of the unit cell. This is, however, limited from the assumption that the sample is 100% crystalline. In reality the crystalline phase of a compound may vary and it is only the crystalline portion of the sample that can be analyzed with a diffraction based technique such as PXRD and MicroED.

MicroED has emerged as a technique for identifying and quantifying the components of complex mixtures, offering high resolution and sensitivity. Crystallization is itself a stereochemically discriminating process and naturally distinguishes between different isomers, including constitutional, conformational, geometric, diastereomer, and enantiomer isomers, based on their crystal structure properties rather than chemical properties. The structure of unknown well diffracting compounds may be obtained with the lower limit of a few picograms of a sample consisting of a few nano sized crystals only, independent of further constituents of the sample. Even without solving the full crystal structure, the unit cell parameters and space group can be used as a signature of the crystal structure and its content. This means that most compounds can be distinguished by their unit cells, making compositional analysis of crystalline samples possible without collecting a full set of crystallographic data. Because determining the unit cell parameters is one of the first steps in structure determination and does not depend on solving the phase problem or collecting high-resolution data, this approach can be used even when the diffraction qualities of the compounds are limited. Relative to the comparative examples discussed above (e.g., PXRD), MicroED provides for faster and higher-quality analyses.

High throughput automation has applications in structural biology methods, particularly in MicroED, where multiple data sets are often required. Autonomous data collection reduces manual labor, increases instrument usage, and allows larger data sets to be collected in the same timeframe, which can reduce the use of scarce computing/processing resources. This is especially important when many data sets must be merged to achieve higher completeness or when crystals are oriented preferentially on the grid. Additionally, studies have shown that automated approaches are effective in analyzing multiple phase systems and distinguishing different crystal forms. For instance, Wang et al. demonstrated the automated analysis of two zeolites using SerialRED for rotation electron diffraction. Smeets et al. used Instamatic to determine two structures from four phases in a multiple phase system. Jones et al. distinguished four natural products from a heterogeneous powder mixture, while Ge et al. identified two different zeolitic-imidazolate frameworks from phase mixtures. Broadhurst et al. identified four forms of carbamazepine based on their unit-cell dimensions and processed them to structures of the respective forms. Luo et al. automatically examined hundreds of crystals using SerialRED and identified five similar zeolite phases, and Sasaki et al. found two synthesized phases using SerialEM in diffraction mode, one of which led to a MicroED structure. These studies demonstrate the potential of high throughput automation in collecting and analyzing data in MicroED and other structural biology methods, and highlight the ability of automated methods to accelerate the discovery of new crystal forms and improve the understanding of complex material structures.

In accordance with various aspects, the present disclosure sets forth systems and methods for compositional analysis of complex mixtures comprising a number of compounds (including salts, saccharides, and amino acids) per sample at varying percentages. The compounds in each group possess comparable physical and chemical characteristics such as weight, hydrophilicity, and charge distribution, which are distinguishing factors in many analytical methods. Some compounds have the same chemical formula but differ in stereochemistry or constitutional isomers, making separation challenging for comparative analysis methods. A high throughput autonomous MicroED approach was developed to enable identification of all constituents and their relative ratios, mostly with high accuracy of just a few percent of their total composition. To demonstrate the extension of MicroED capabilities using this approach, the method was further applied to an additional complex mixture sample. All the ingredients in a mixture of nine compounds with a wide distribution of amounts per weight in the composition were identified, with the lowest amount of constituents being around 3% by total mass, showing the effectiveness of the systems and methods described herein. The method was also applied to drug formulations of aspirin and acetaminophen tablets. Among other crystalline ingredients, the active ingredients were identified with less than 2% errors in their mass ratios.

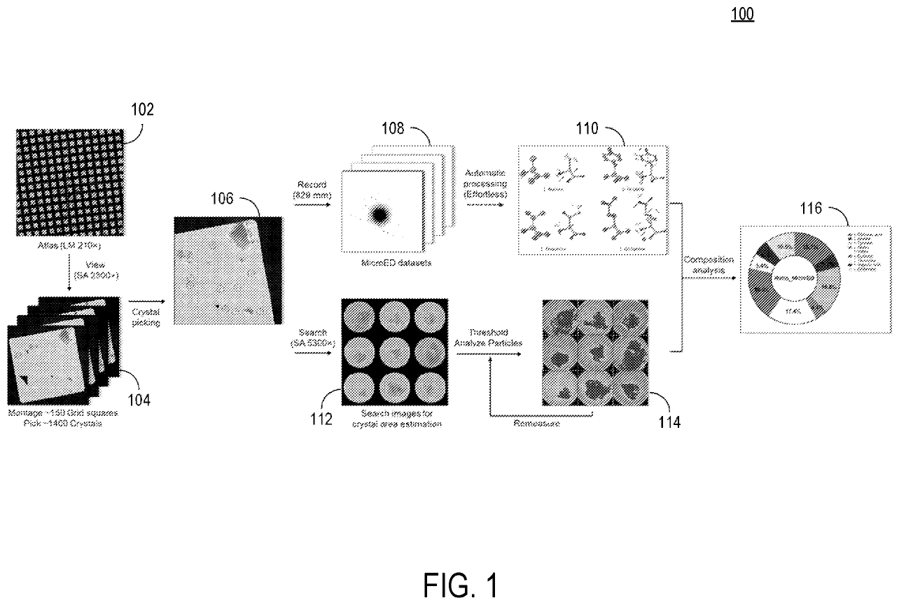

Although automation often requires a significant amount of software development, examples of the approach set forth in the present disclosure utilized widely available software, such as SerialEM and other available X-ray crystallographic processing software. In one example, procedures were developed in SerialEM and coupled to a Python script that was developed to automatically collect and process MicroED data generated from the procedures in real-time. FIG. 1 illustrates an exemplary workflow 100 for autonomous MicroED data collection and processing according to various aspects of the present disclosure. The method includes collecting an atlas montage of the entire grid, followed by medium-magnification montages of the most promising grid squares. The eucentric height is then automatically determined for each grid square and stored with the medium montage maps. The crystal positions are accurately determined from the montage maps and stored as a list of points in SerialEM. Each point in the list is queued for data collection, applying a SerialEM macro to each point. In some examples, the grid includes a plurality of grid squares (for example and without limitation, 50-800) each grid square contains 10 crystals of the appropriate size and thickness, and about 150 medium magnification montages are needed to assemble a data collection of 1500 crystals (although in implementations the data collection may include 20 or more crystals; and in some implementations may include more, and sometimes much more, than 1500 crystals). An example data set of 50° continuous rotation collected at 2°/s takes approximately one minute to collect on the Talos Arctica and over 1000 complete data sets may be collected autonomously overnight.

In FIG. 1, at operation 102 a low-magnification atlas is used to screen the grid containing a mixture of compounds, the grids being further illustrated in operation 104. Suitable grid squares are selected in operation 106 for medium-magnification montages. Crystals are then chosen and added to a list for data collection. Next, a continuous rotation MicroED movie is obtained in operation 108, and an image of the crystal is autonomously collected (e.g., using SerialEM) in operation 110. Data is then processed in real-time, where crystal images are searched for crystal volume estimation in operation 112 for compositional analysis, where threshold analysis particles are remeasured (if necessary) in operation 114), and a composition analysis is output from the workflow 100 in operation 116. Workflow 100 allows for efficient and autonomous MicroED data collection and processing of thousands of data sets of a period of hours, reducing the need for human intervention and saving researchers' time and computing resources while producing high quality data.

The workflow 100 was used to determine the components in complex mixtures using several different mixtures as proof of principle. Mixture A contained several inorganic salts while mixtures B and C contained several saccharides and amino acids, respectively. These mixtures were analyzed using the pipeline to identify all their components. In addition to the manually prepared heterogeneous mixtures, the components from the aspirin and acetaminophen mixtures which were ground from the commercial drugs were analyzed. The crystalline ingredients were successfully identified and observed with promising consistency to the values listed in the drug information.

### Autonomous High-Throughput Data Collection and Processing

To demonstrate the efficacy and advantages of the present disclosure, a Python script was developed to process data generated from the autonomous approach in real time. This script was designed to keep up with the speed of data acquisition and to customize the processing for MicroED data recorded using the Falcon detector and the Talos Arctica. The pipeline includes converting the .mrc image file format to smv format, and running XDS, XSCALE, and XDSCONV to process, merge, and convert the data to SHELX .hkl file format. The pipeline may use a brute force approach to evaluate a combination of frequently used input parameters as the best statistics may not be found with exactly the same XDS input parameters. The best solution is selected based on the results of XSCALE, and the pipeline systematically explores combinations of data sets to merge, and finally uses a scoring function to select the top merged data sets which are suggested to the user. This approach resulted in a robust pipeline to process MicroED data from various samples without the need to select input parameters prior to processing or assuming similarity among data sets.

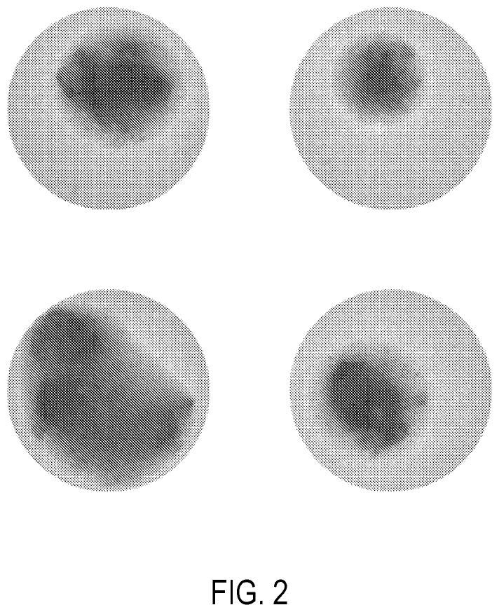

Mixture A was prepared with varying amounts of salts ranging from 3.7 to 29.3 mg, totaling to 112.0 mg, as shown in Table 1. The mixture composed of Sodium bicarbonate (14.4% v/v), Sodium sulfate (21.3% v/v), Potassium sulfate (14.2% v/v), Magnesium sulfate heptahydrate (6.7% v/v), Sodium chloride (5.4% v/v), Calcium gluconate (5.9% v/v), Sodium citrate dihydrate (15.4% v/v), Calcium acetate monohydrate (4.7% v/v), and Magnesium acetate tetrahydrate (12.0% v/v). When this mixture was loaded to the microscope, several crystals appeared “melted” as their edges were not sharp, suggesting that somehow in the process hydration may have occurred which may lead to a loss in crystal order and resolution. These are shown in FIG. 2. In the images shown in FIG. 2, there are visually two areas of different contrast: a darker particle-shaped inner area surrounded by a lighter drop-like outer area. Hydration may have happened in the test tube prior to grid preparation, and/or the crystals may have partially decomposed before being frozen in the microscope, resulting in poor diffraction. Several crystals were also observed clumped together, so they were not suitable for analysis. Regardless of these concerns, 1961 crystals were selected for automatic diffraction and out of these only 913 (47%) delivered sufficiently good data for identification using unit cell parameters as discriminators (see Table 1). Because the images of the crystals were also recorded, it was possible go back and look at the morphologies of the crystals that did not diffract. These issues with problematic crystals may be reduced or eliminated by careful sample preparation. Despite this concern it was possible to identify all components in the mixture and even to identify the constituent with lowest percentage. Calcium acetate monohydrate which was added at ~4.7% in the input was identified in 40 crystals corresponding to a 4.4% counting ratio.

TABLE 1

| | Density | Mass | No. |
|---|---|---|---|
| Mixture A Component | (g/cm³) | (mg) | crystals |
| Sodium bicarbonate | 2.20 | 16.32 | 19 |
| Sodium sulfate | 2.67 | 29.31 | 95 |
| Potassium sulfate | 2.66 | 19.49 | 122 |
| Magnesium sulfate heptahydrate | 2.66 | 9.14 | 19 |
| Sodium chloride | 2.17 | 6.07 | 5 |
| Calcium gluconate | 1.68 | 5.14 | 4 |
| Sodium citrate dihydrate | 1.76 | 13.92 | 136 |
| Calcium acetate monohydrate | 1.50 | 3.66 | 40 |
| Magnesium acetate tetrahydrate | 1.45 | 8.93 | 126 |

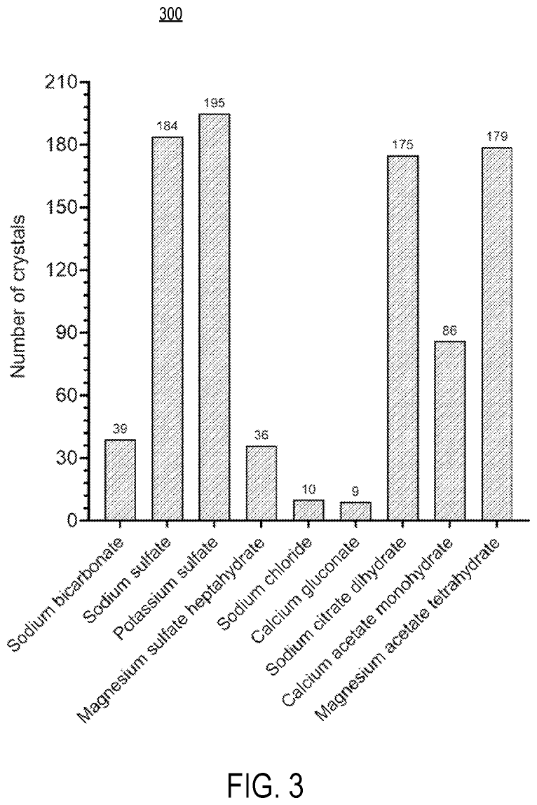

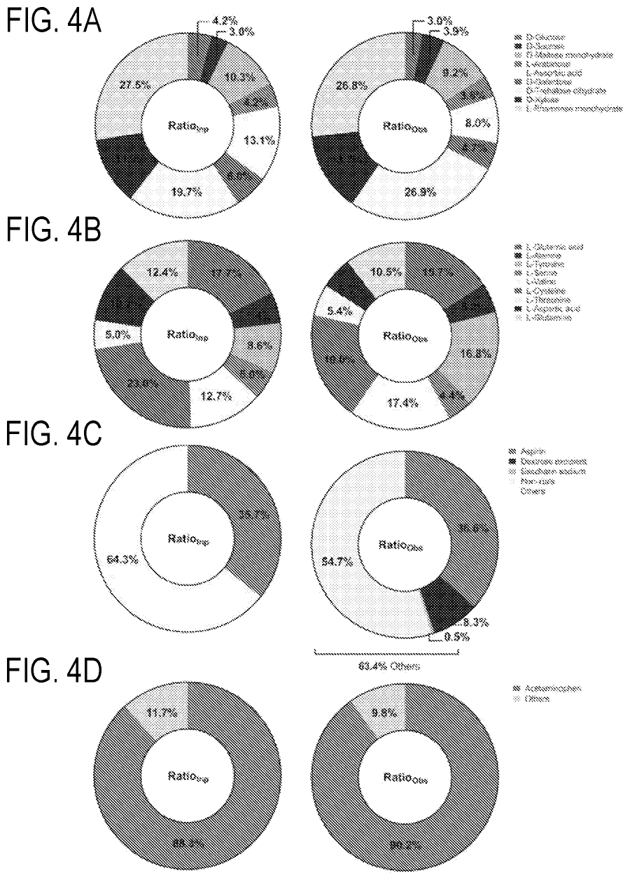

FIG. 3 shows a bar graph 300 illustrating the numbers of crystals identified for each composition in mixture A. Despite the smaller number of datasets used (913 in total), all compounds could be identified including the compounds with the lowest relative mass of 3% of the total weight of the composition. FIGS. 4A-4D are graphs illustrating the compositional analysis of mixture B, mixture C, the aspirin tablet, and the acetaminophen tablet, respectively, according to the methods described herein. The compositional analysis of the mixtures shows the relative volumes in the actual mixture compared to the analysis based on autonomous MicroED data. In FIGS. 4A-4D, the Ratio_{Inp} represents the volume ratio (%) for each component in the original composition, while the Ratio_{Obs} was calculated by dividing the ratio of the total area of one component to the total area of all crystals in the MicroED analysis.

In the second set of mixed compounds, a total of nine different saccharides, along with a similar scaffold (L-Ascorbic acid), were weighed in with relative amounts ranging from 3.0 to 27.5% (Mixture B), as shown in Table 2. The components of this mixture were: D-Glucose (4.2% v/v), D-Sucrose (3% v/v), D-Maltose monohydrate (10.3% v/v), L-Arabinose (4.2% v/v), L-Ascorbic acid (13.1% v/v), D-Galactose (6% v/v), D-Trehalose dihydrate (19.7% v/v), D-Xylose (11.9% v/v) and L-Rhamnose monohydrate (27.5% v/v). Separating some of the saccharides using size or affinity chromatography based on hydrophobicity or charge is challenging using comparative methods due to their chemical similarities. For instance, four of the saccharides, L-Arabinose, D-Xylose, D-Galactose, and D-Glucose, are pairwise chemically identical and differ only in the position of a hydroxyl group (diastereomers), which requires stereochemical separation. Trehalose and Maltose are also chemically identical, consisting of two glucose entities and differing only at the glycosidic linkage sites of the two pyranose rings. Similarly, sucrose is only one methylene group larger than maltose, which is similar in size. However, the crystallographic unit cells for all of these compounds are distinct from each other and distinguished in MicroED, making crystallographic analysis of chemically identical but stereochemically different compounds possible.

TABLE 2

| | Density | Mass | Volume | |
|---|---|---|---|---|
| Mixture B Component | (g/cm³) | (mg) | (mm³) | % v/v |
| D-Glucose | 1.56 | 5.31 | 3.40 | 4.2 |
| D-Sucrose | 1.59 | 3.88 | 2.44 | 3.0 |
| D-Maltose monohydrate | 1.77 | 14.70 | 8.31 | 10.3 |
| L-Arabinose | 1.59 | 5.42 | 3.41 | 4.2 |
| L-Ascorbic acid | 1.65 | 17.55 | 10.64 | 13.1 |
| D-Galactose | 1.50 | 7.30 | 4.87 | 6.0 |
| D-Trehalose dihydrate | 1.76 | 28.05 | 15.94 | 19.7 |
| D-Xylose | 1.53 | 14.73 | 9.63 | 11.9 |
| L-Rhamnose monohydrate | 1.47 | 32.73 | 22.27 | 27.5 |

For the saccharides mixture (B), there were 1374 data sets autonomously collected, of which 1000 (73%) were successfully identified as having one of the unit cells in the mixture. This was a substantial increase in success rate from 47% in mixture A to 73% in mixture B. As with Mixture A, the remaining 27% either did not diffract well possibly due to hydration, or originated from crystal clumps or were not crystalline to begin with. Importantly, all components of the mixture could be identified and for most the composition was determined within a 2% error. Only two compounds, L-Ascorbic acid and D-Trehalose dihydrate, demonstrated larger errors of 5.1% and 7.2%, respectively. Without wishing to be bound by any particular theory of operation, it is believed that occasionally crystals have a tendency to lose their diffraction properties upon grinding. Deviations in the ratios of the constituents of the mixtures analyzed may come from differences in the size distributions of the sample crystals after grinding, and damages to the crystals during sample preparation. It is also possible that materials adhere differently to the grid, and this could also lead to minor errors. Because only the area is estimated and not the volume of the crystalline grains, this may introduce uncertainty. It was noted, however, that the difference between the crystal counting results and the area-corrected results is small and the area estimation did not substantially influence the result (<2%). Overall, using MicroED in an automatic setting, only one grid containing the mixture was prepared and mounted, from which after processing the relative ratios could be derived. FIG. 4A and Table 3 illustrate these results. In mixture B, the relative volumes of the compounds were found to be consistent between the original composition and the MicroED analysis, with only slight deviations for some compounds.

TABLE 3

| | No. | Area crystals | Ratio_{Obs} |
|---|---|---|---|
| Mixture B Component | crystals | (μm²) | (%) |
| D-Glucose | 32 | 8.10 | 3.0 |
| D-Sucrose | 31 | 10.71 | 3.9 |
| D-Maltose monohydrate | 92 | 25.13 | 9.2 |
| L-Arabinose | 33 | 10.20 | 3.8 |
| L-Ascorbic acid | 74 | 21.68 | 8.0 |
| D-Galactose | 52 | 12.70 | 4.7 |
| D-Trehalose dihydrate | 255 | 73.19 | 26.9 |
| D-Xylose | 145 | 37.34 | 13.7 |
| L-Rhamnose monohydrate | 286 | 72.80 | 26.8 |

In Table 3, “No. Crystals” represents the number of crystals identified for each component. In determining the area of crystals, the crystal areas were converted from pixels² to μm² using the pixel sizes and magnification. The observed ratio Ratio_{Obs} was calculated by dividing the ratio of the total area of one component to the total area of all crystals.

The approach was next applied to a third class of compounds—amino acids. Mixture C, described in Table 4, contained L-Glutamic acid (17.7% v/v), L-Alanine (5.4% v/v), L-Tyrosine (8.6% v/v), L-Serine (5% v/v), L-Valine (12.7% v/v), L-Cysteine (23% v/v), L-Threonine (5% v/v), L-Aspartic acid (10.2% v/v), L-Glutamine (12.4% v/v). 1401 crystals were selected for autonomous MicroED analyses and this time the success rate was even higher, as 1121 out of 1401 (80%) crystals were identified. The area-corrected ratios of compounds in mixture C were found to be relatively similar to the ratios obtained by counting the number of crystals, with most ratios within 2% in relative amounts. However, for L-Tyrosine, L-Aspartic acid, and L-Glutamine, which had larger relative errors, the area corrected ratios tended to be better than the uncorrected ones, particularly for L-Tyrosine, where the overestimated ratio dropped from 10.6% to 8.2% after area correction. The estimated ratios are close to the weighed-in ratios in this analysis (see FIG. 4B and Table 5). These results suggest that sample preparation and the material used for grid preparation are key determinants. It is possible that the amorphous carbon support used in these experiments is more suitable for biological material like amino acids.

TABLE 4

| | Density | Mass | Volume | |
|---|---|---|---|---|
| Mixture C Component | (g/cm³) | (mg) | (mm³) | % v/v |
| L-Glutamic acid | 1.54 | 22.72 | 14.75 | 17.7 |
| L-Alanine | 1.43 | 6.45 | 4.51 | 5.4 |
| L-Tyrosine | 1.46 | 10.47 | 7.17 | 8.6 |
| L-Serine | 1.60 | 6.62 | 4.14 | 5.0 |
| L-Valine | 1.23 | 13.04 | 10.60 | 12.7 |
| L-Cysteine | 1.30 | 24.95 | 19.19 | 23.0 |
| L-Threonine | 1.31 | 5.50 | 4.20 | 5.0 |
| L-Aspartic acid | 1.66 | 14.11 | 8.50 | 10.2 |
| L-Glutamine | 1.36 | 14.13 | 10.39 | 12.4 |

TABLE 5

| Mixture C | | | |
|---|---|---|---|
| Component | No. crystals | Area crystals (μm²) | Ratio_{Obs} (%) |
| L-Glutamic acid | 172 | 45.66 | 15.7 |
| L-Alanine | 53 | 15.08 | 5.2 |
| L-Tyrosine | 215 | 48.85 | 16.8 |
| L-Serine | 51 | 12.66 | 4.4 |
| L-Valine | 197 | 50.41 | 17.4 |
| L-Cysteine | 207 | 55.12 | 19.0 |
| L-Threonine | 68 | 15.78 | 5.4 |
| L-Aspartic acid | 57 | 16.04 | 5.5 |
| L-Glutamine | 101 | 45.66 | 10.5 |

In Table 5, “No. Crystals” represents the number of crystals identified for each component. In determining the area of crystals, the crystal areas were converted from pixels² to μm² using the pixel sizes and magnification. The observed ratio Ratio_{Obs} was calculated by dividing the ratio of the total area of one component to the total area of all crystals.

As a further assessment of the general applicability of the method, the workflow 100 was applied on two commercially available drug tablets: formulations of aspirin and acetaminophen. Drug formulations may contain a variety of non-active agents such as binders, disintegrants, sugar and wax. They may not be in a crystalline state and could therefore potentially be problematic for a method based on the crystallinity of the compounds. In fact, the aspirin formulation contains only 35.7% (w/w %) of the active ingredient in a formulation of mostly non-active components, as shown in Table 6. A few of the non-active components were crystalline and their unit cell dimensions were found in the analysis. A substantial part, however, consists of ingredients for which the crystalline status is undeclared, such as for corn starch, carnauba wax, FD&C yellow no. 6 aluminum lake and flavors. For starch alone a partial crystallinity is often reported as well as several crystalline forms, making a complete analysis of this part of the formulation difficult. In order to extract valid ratios, therefore, the number of crystals with a unit cell corresponding to the active ingredient were compared to the total amount of well diffracting as well as non-diffracting grains selected. The remaining smaller group for which some diffraction was recorded but the diffraction was not enough for unit cell determination, and therefore was considered as of undefined status and left out of the calculations as a whole. This was necessary as a conclusion as to whether a weakly diffracting grain should be considered originating from a partially disordered crystalline grain or from an anticipated but partially organized amorphous phase may not be possible. If such a grain originates from an anticipated amorphous phase, which is to some extent crystalline but not enough for determining the unit cell, for instance, the crystalline portion could be overestimated. The analysis for aspirin, therefore, resulted in 36.6% of the diffracting crystals belonging to the unit cell of aspirin and smaller fractions, 8.3% and 0.5% was identified as dextrose and saccharin respectively (FIG. 4C, Table 7). Considering the large amount of non-diffracting grains, the error of less than one percent is surprisingly accurate (see Table 7).

TABLE 6

| Aspirin Tablet Component | Mass (mg) | % w/w |
|---|---|---|
| Aspirin | 81 | 35.7 |
| Others | 146.17 | 64.3 |

TABLE 7

| Aspirin Tablet | | | |
|---|---|---|---|
| Component | No. crystals | Area crystals (μm²) | Ratio_{Obs} (%) |
| Aspirin | 472 | 418.56 | 36.6 |
| Dextrose excipient | 103 | 94.82 | 8.3 |
| Saccharin sodium | 4 | 5.23 | 0.5 |
| Non-crystals | 715 | 626.00 | 54.7 |

In Table 6, the ingredients composition in the aspirin tablet was obtained from the “Drug facts” declared on the package. The aspirin tablet contains 81 mg aspirin (active ingredient) and others (inactive ingredients) including corn starch, dextrose excipient, FD&C yellow no. 6 aluminum lake, flavors, saccharin sodium. In Table 7, “No. Crystals” represents the number of crystals identified for each component. In determining the area of crystals, the crystal areas were converted from pixels² to μm² using the pixel sizes and magnification. The observed ratio Ratio_{Obs} was calculated by dividing the ratio of the total area of one component to the total area of all crystals. Aspirin contains two polymorphs of aspirin; and dextrose excipient contains crystals of α-D-glucose, β-D-glucose, and α-D-glucose monohydrate.

For acetaminophen, on the other hand, the largest portion in the formulation, 88.3% (w/w %, Table 8), consists of the active ingredient. A similar analysis was done for this sample which resulted in an estimated ratio of 90.2% of the active ingredient (FIG. 4D, Table 9). No other crystalline grains were found within this formulation.

TABLE 8

| Acetaminophen Tablet Component | Mass (mg) | % w/w |
|---|---|---|
| Acetaminophen | 500 | 88.3 |
| Others | 66.26 | 11.7 |

TABLE 9

| Acetaminophen Tablet | | Area of crystals | |
|---|---|---|---|
| Component | No. crystals | (μm²) | Ratio_{Obs} (%) |
| Acetaminophen | 652 | 667.72 | 90.2 |
| Others | 56 | 72.82 | 9.8 |

In Table 8, the ingredients composition in the acetaminophen tablet was obtained from the “Drug facts” declared on the package. The acetaminophen tablet contains 500 mg acetaminophen (active ingredient) and others (inactive ingredients) including carnauba wax, hypromellose, polyethylene glycol, povidone, pregelatinized starch, stearic acid. The acetaminophen tablet may contain one or more of corn starch, croscarmellose sodium, and sodium starch glycolate. In Table 9, “No. Crystals” represents the number of crystals identified for each component. In determining the area of crystals, the crystal areas were converted from pixels² to μm² using the pixel sizes and magnification. The observed ratio Ratio_{Obs} was calculated by dividing the ratio of the total area of one component to the total area of all crystals. Overall, the results illustrated in FIGS. 4A-4D demonstrate the feasibility and accuracy of using MicroED data for composition analysis of complex mixtures.

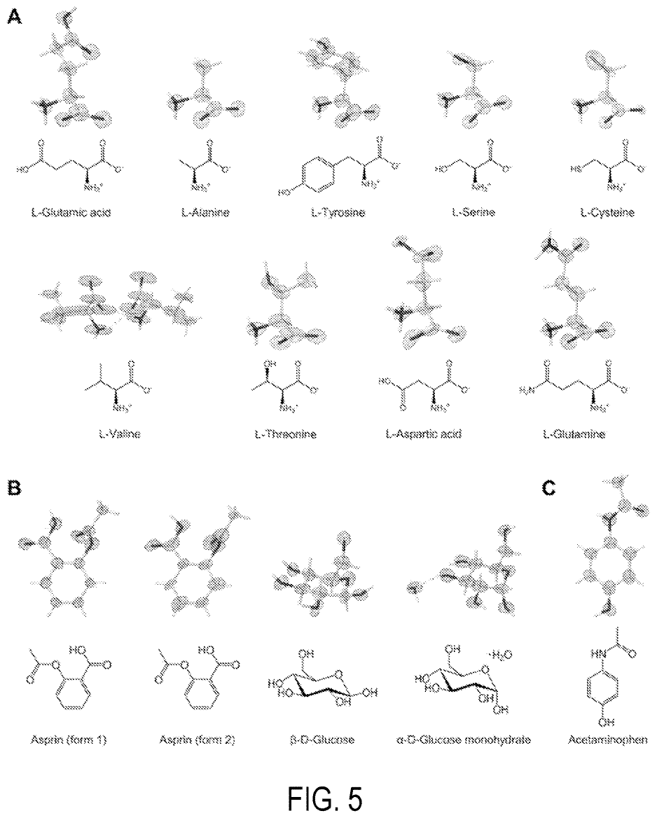

Although only a small wedge of data is collected from each crystal, data sets can be merged and structures solved as needed. This approach was applied to data sets from mixture C, aspirin tablets, and acetaminophen tablets, and it was possible to solve all components to subatomic resolution, with completeness levels greater than 80% and more than 2000 observed reflections in most cases even for these small molecules. FIG. 5 illustrates the component structures for Mixture C, the Aspirin tablet, and the Acetaminophen tablet. Tables 10-12 illustrate the MicroED refinement statistics for the crystal structures solved from the autonomous SerialEM data collection. Structures were solved ab initio by SHELXT and refined by SHELXL at 0.75 Å, following automatic processing by the implemented Python script. The blue meshes in FIG. 5 are 2Fₒ-F_{c} electrostatic potential maps, green meshes are Fₒ-F_{c} electrostatic potential maps, the position of hydrogen atoms are marked with black arrows (presented by Olex2). The electrostatic potential level for each compound in 2Fₒ-F_{c} and Fₒ-F_{c} maps are as listed: L-Glutamic acid (0.85 and 0.21 e·Å⁻³), L-Alanine (0.85 and 0.20 e Å⁻³), L-Tyrosine (0.85 and 0.15 e·Å⁻³), L-Serine (0.85 and 0.20 e·Å⁻³), L-Valine (0.46 and 0.09 e·Å⁻³), L-Cysteine (0.85 and 0.31 e·Å⁻³), L-Threonine (0.85 and 0.16 e Å⁻³), L-Aspartic acid (0.38 and 0.11 e·Å⁻³), L-Glutamine (0.85 and 0.11 e·Å⁻³), aspirin form 1 (0.74e·Å⁻³), aspirin form 2, (1.31e Å⁻³), β-D-Glucose (1.10 e·Å⁻³); α-D-Glucose monohydrate (1.21 e·Å⁻³), acetaminophen (1.04 e·Å⁻³). In Tables 10-12, “completeness” refers to the completeness of the merged datasets by XSCALE.

TABLE 10

| Mixture C | No. Merged | Res. | Completeness | No. Unique | No. Observed | R₁ |
|---|---|---|---|---|---|---|
| Component | dataset | (Å) | (%) | Reflections | Reflections | (%) |
| L-Glutamic acid | 2 | 0.75 | 89.4 | 838 | 3120 | 18.2 |
| L-Alanine | 2 | 0.75 | 93.3 | 596 | 2050 | 11.5 |
| L-Tyrosine | 3 | 0.75 | 89.8 | 1093 | 6240 | 12.6 |
| L-Serine | 2 | 0.75 | 82.2 | 559 | 2245 | 15.5 |
| L-Valine | 2 | 0.75 | 49.4 | 803 | 2936 | 15.2 |
| L-Cysteine | 2 | 0.75 | 92.4 | 731 | 2676 | 17.2 |
| L-Threonine | 2 | 0.75 | 83.0 | 676 | 2709 | 13.2 |
| L-Aspartic acid | 4 | 0.75 | 91.4 | 647 | 2663 | 21.5 |
| L-Glutamine | 3 | 0.75 | 98.2 | 915 | 4447 | 13.5 |

TABLE 11

| Aspirin | No. | | Complete- | No. | No. | |
|---|---|---|---|---|---|---|
| Tablet | Merged | Res | ness | Unique | Observed | R₁ |
| Component | dataset | (Å) | (%) | Reflections | Reflections | (%) |
| Aspirin (form 1) | 4 | 0.72 | 83.3 | 1855 | 14444 | 19.6 |
| Aspirin (form 2) | 3 | 0.92 | 85.2 | 958 | 5314 | 21.7 |
| β-D-Glucose | 2 | 0.70 | 94.5 | 1280 | 7973 | 19.9 |
| α-D-Glucose | 4 | 0.71 | 81.6 | 1147 | 8443 | 15.2 |
| monohydrate | | | | | | |

TABLE 12

| Acetaminophen | No. | | Complete- | No. | No. | |
|---|---|---|---|---|---|---|
| Tablet | Merged | Res. | ness | Unique | Observed | R₁ |
| Component | dataset | (Å) | (%) | Reflections | Reflections | (%) |
| Acetaminophen | 3 | 0.70 | 96.0 | 2088 | 10643 | 19.6 |

Once the data was collected and processed, unit cell parameters were used for chemical identification. Using the unit cell parameters to identify the crystal structures has several advantages over solving structures for all data sets. First, it allows for shorter rotation wedges to be collected for each data set, increasing the number of data sets that can be collected within a set time. Second, the unit cell parameters are easier to extract than solving structures for all data sets. This is particularly advantageous when collecting data on a large number of crystals that vary in size and diffraction quality, as some data sets may not be good enough for proper structure determination but sufficiently good for accurate unit cell and symmetry determination. Finally, using unit cell parameters to identify structures results in a higher accuracy of assignments, which affects the identification of compounds present in small amounts in the mixture. Overall, this approach allowed for the successful identification of all salts, including those present in the lowest amounts in the mixture.

To validate the approach, an analysis was performed to assess the risk of incorrectly assigning a dataset to the wrong crystal structure based on similarities in the unit cell parameters. Narrow windows were established around the published values for the unit cell dimensions and angles, allowing for deviations of up to 1 Å and 10°, respectively. These restrictions were compared with the actual distributions of unit cell parameters in the sample mixtures, and the results showed that the actual parameters fell well within the allowed values. Furthermore, no overlap was found in the absolute values and combinations of unit cell parameters for any two crystal forms, indicating that the proper crystal structure could be unambiguously determined for all the compounds involved in the experiments. Overall, the analysis confirmed that the unit cell parameters were a unique and easy-to-compare property for the crystal structures, and accurately identifying the compounds in the mixtures.

In addition, to validate the crystal selection process, the diffraction quality of the selected crystals was analyzed. The distribution of resolution and completeness of the diffraction patterns were compared between the different compounds and showed no significant difference. This is shown in Table 1, which shows MicroED refinement statistics for Mixture C, which as noted above is a mixture containing amino acids for which the structures were solved from the automatic data collection using SerialEM. Moreover, the refined Ri factors reflect an overall excellent MicroED data quality with no significant difference between the compounds. These results suggest that the crystal selection process did not introduce significant bias in the diffraction quality or thermal motion of the selected crystals. For each crystal, the volume of the diffracting part was estimated by multiplying the area of the crystal by a thickness factor that accounts for the variation in crystal thickness. This thickness factor was calibrated by comparing the electron transmission through several crystals of known thickness, and it was assumed to be constant for all crystals within the selected size range. The estimated crystal volumes were found to be similar across all compounds, indicating that the sample preparation process did not result in a bias towards a particular compound due to differences in crystal hardness or brittleness.

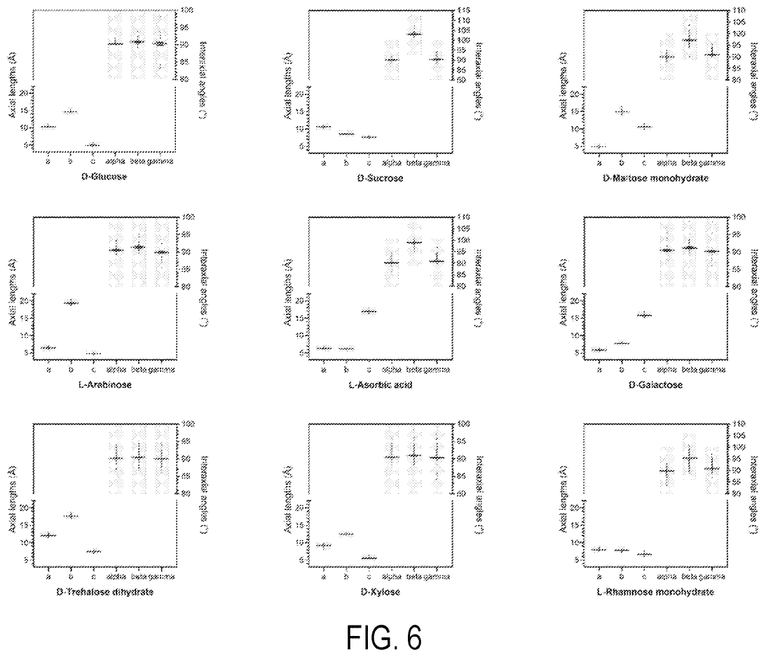

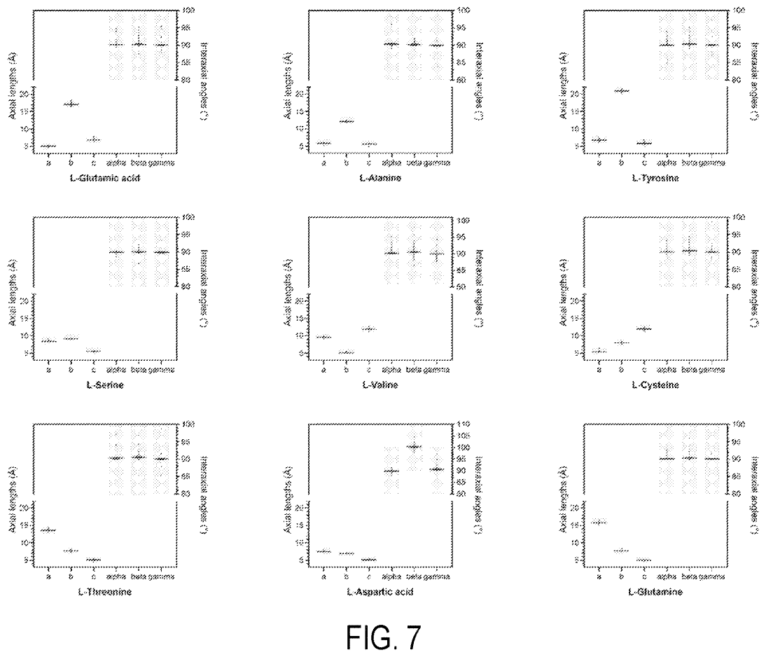

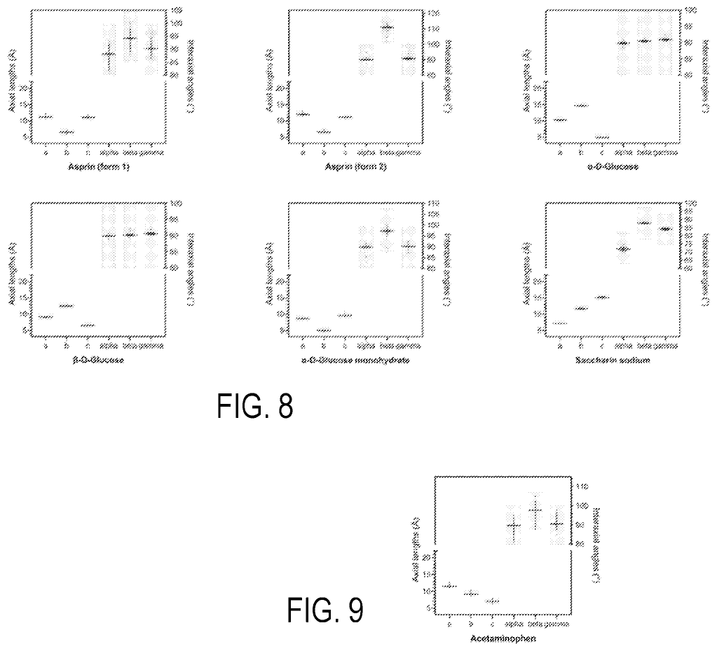

FIG. 6 is a series of graphs illustrating the unit cell parameters from processing in mixture B. FIG. 7 is a series of graphs illustrating unit cell parameters from processing in mixture C. FIG. 8 is a series of graphs illustrating unit cell parameters from processing in the aspirin tablet. FIG. 9 is a series of graphs illustrating unit cell parameters from processing in the acetaminophen tablet. In each of FIGS. 6-9, each graph shows unit cell parameters plotted as red dots, standard deviations plotted as black lines, and the tolerance bars are shown by blue and green boxes. The experimental unit cell parameters fall well within the selected tolerance limits, which ensures an accurate cell determination, and there is no overlap of the six unit cell parameters between the space groups and the structure assignments were conclusive. Tables 13-17 show reference unit cell parameters in mixtures A-C, the aspirin tablet, and the acetaminophen tablet, respectively.

TABLE 13

| | CSD/ICSD | | | | | | | |
|---|---|---|---|---|---|---|---|---|
| Mixture A Component | entry | Space group | a | b | c | α | β | γ |
| Sodium bicarbonate | 26933 | P 1 2₁/n 1(14) | 7.51 | 9.70 | 3.53 | 90.0 | 93.0 | 90.0 |
| Sodium sulfate | 136895 | F d d d (70) | 5.86 | 12.31 | 9.82 | 90.0 | 90.0 | 90.0 |
| Potassium sulfate | 79777 | P m c n (62) | 5.75 | 10.04 | 7.45 | 90.0 | 90.0 | 90.0 |
| Magnesium sulfate heptahydrate | 16111 | P 2₁ 2₁ 2₁ (19) | 11.87 | 12.00 | 6.86 | 90.0 | 90.0 | 90.0 |
| Sodium chloride | 29929 | F m −3 m (225) | 5.63 | 5.63 | 5.63 | 90.0 | 90.0 | 90.0 |
| Calcium gluconate | YUXDED | C 2 (5) | 15.87 | 5.88 | 9.16 | 90.0 | 99.6 | 90.0 |
| Sodium citrate dihydrate | UMOGAE | C 2/c (15) | 15.70 | 12.50 | 11.28 | 90.0 | 103.6 | 90.0 |
| Calcium acetate monohydrate | CEJLIM | P-1 (2) | 6.75 | 11.08 | 11.78 | 116.0 | 92.0 | 97.0 |
| Magnesium acetate tetrahydrate | FELGUY01 | P 2₁/c (14) | 4.81 | 11.99 | 8.55 | 90.0 | 95.4 | 90.0 |

TABLE 14

| | CSD/ICSD | | | | | | | |
|---|---|---|---|---|---|---|---|---|
| Mixture B Component | entry | Space group | a | b | c | α | β | γ |
| D-Glucose | GLUCSA | P 2₁ 2₁ 2₁ (19) | 10.36 | 14.84 | 4.97 | 90.0 | 90.0 | 90.0 |
| D-Sucrose | SUCROS03 | P 2₁ (4) | 10.86 | 8.70 | 7.76 | 90.0 | 103.0 | 90.0 |
| D-Maltose monohydrate | MALTOS11 | P 2₁ (4) | 4.87 | 15.08 | 10.70 | 90.0 | 97.1 | 90.0 |
| L-Arabinose | ABINOS01 | P 2₁ 2₁ 2₁ (19) | 6.51 | 19.45 | 4.84 | 90.0 | 90.0 | 90.0 |
| L-Ascorbic acid | LASCAC12 | P 2₁ (4) | 6.39 | 6.26 | 17.13 | 90.0 | 99.4 | 90.0 |
| D-Galactose | ADGALA01 | P 2₁ 2₁ 2₁ (19) | 5.94 | 7.87 | 15.80 | 90.0 | 90.0 | 90.0 |
| D-Trehalose dihydrate | TREHAL01 | P 2₁ 2₁ 2₁ (19) | 12.23 | 17.89 | 7.60 | 90.0 | 90.0 | 90.0 |
| D-Xylose | ZZZSEA | P 2₁ 2₁ 2₁ (19) | 9.10 | 12.10 | 5.60 | 90.0 | 90.0 | 90.0 |
| L-Rhamnose monohydrate | RHAMAH01 | P 2₁ (4) | 7.91 | 7.92 | 6.67 | 90.0 | 95.6 | 90.0 |

TABLE 15

| Mixture C | CSD/ICSD | | | | | | | |
|---|---|---|---|---|---|---|---|---|
| Component | entry | Space group | a | b | c | α | β | γ |
| L-Glutamic acid | LGLUAC | P 2₁ 2₁ 2₁ (19) | 5.17 | 17.34 | 6.95 | 90.0 | 90.0 | 90.0 |
| L-Alanine | LALNIN | P 2₁ 2₁ 2₁ (19) | 6.03 | 12.34 | 5.78 | 90.0 | 90.0 | 90.0 |
| L-Tyrosine | LTYROS10 | P 2₁ 2₁ 2₁ (19) | 6.91 | 21.12 | 5.83 | 90.0 | 90.0 | 90.0 |
| L-Serine | LSERIN01 | P 2₁ 2₁ 2₁ (19) | 8.60 | 9.35 | 5.62 | 90.0 | 90.0 | 90.0 |
| L-Valine | LVALIN | P 2₁ (4) | 9.71 | 5.27 | 12.06 | 90.0 | 90.8 | 90.0 |
| L-Cysteine | LCYSTN34 | P 2₁ 2₁ 2₁ (19) | 5.42 | 8.11 | 12.15 | 90.0 | 90.0 | 90.0 |
| L-Threonine | LTHREO | P 2₁ 2₁ 2₁ (19) | 13.61 | 7.74 | 5.14 | 90.0 | 90.0 | 90.0 |
| L-Aspartic acid | LASPRT | P 2₁ (4) | 7.62 | 6.98 | 5.14 | 90.0 | 99.8 | 90.0 |
| L-Glutamine | GLUTAM | P 2₁ 2₁ 2₁ (19) | 16.01 | 7.76 | 5.10 | 90.0 | 90.0 | 90.0 |

TABLE 16

| Aspirin Tablet | CSD/ICSD | | | | | | | |
|---|---|---|---|---|---|---|---|---|
| Component | entry | Space group | a | b | c | α | β | γ |
| Asprin (form 1) | ACSALA01 | P 2₁/c (14) | 11.43 | 6.59 | 11.4 | 90.0 | 95.7 | 90.0 |
| Asprin (form 2) | ACSALA15 | P 2₁/c (14) | 12.15 | 6.51 | 11.37 | 90.0 | 111.6 | 90.0 |
| α-D-Glucose | GLUCSA | P 2₁ 2₁ 2₁ (19) | 10.36 | 14.84 | 4.97 | 90.0 | 90.0 | 90.0 |
| β-D-Glucose | GLUCSE | P 2₁ 2₁ 2₁ (19) | 9.29 | 12.65 | 6.70 | 90.0 | 90.0 | 90.0 |
| α-D-Glucose monohydrate | GLUCMH11 | P 2₁ (4) | 8.80 | 5.09 | 9.71 | 90.0 | 97.7 | 90.0 |
| Saccharin sodium | MGSACD11 | P-1 (2) | 7.27 | 11.60 | 15.13 | 72.5 | 87.7 | 84.0 |

TABLE 17

| Acetaminophen | CSD/ICSD | | | | | | | |
|---|---|---|---|---|---|---|---|---|
| Tablet Component | entry | Space group | a | b | c | α | β | γ |
| Acetaminophen | HXACAN03 | P 2₁/n | 11.73 | 9.38 | 7.11 | 90.0 | 97.5 | 90.0 |

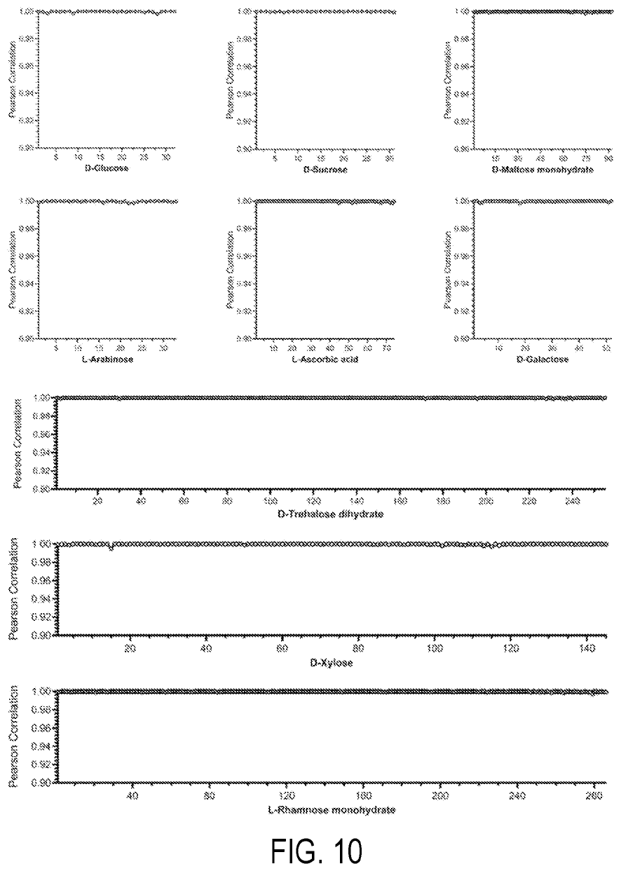

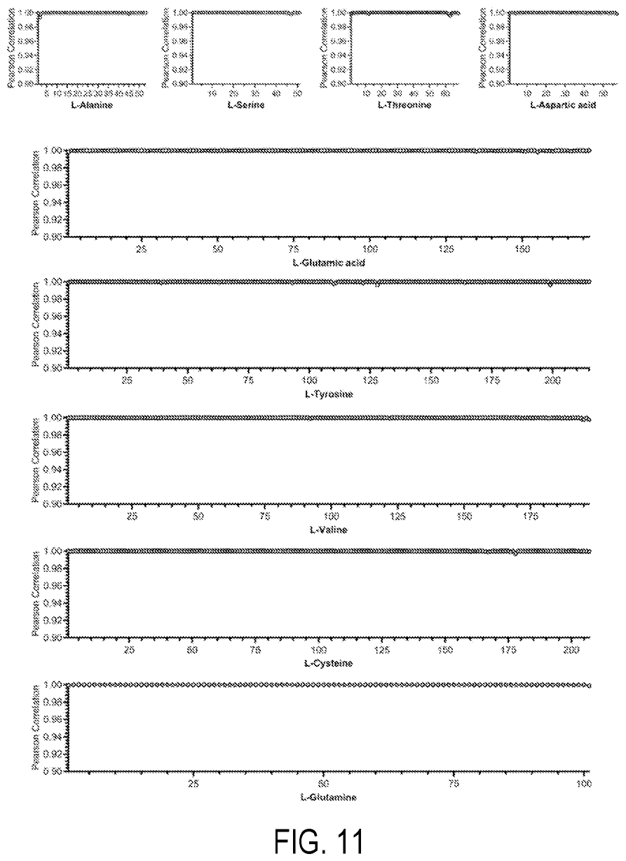

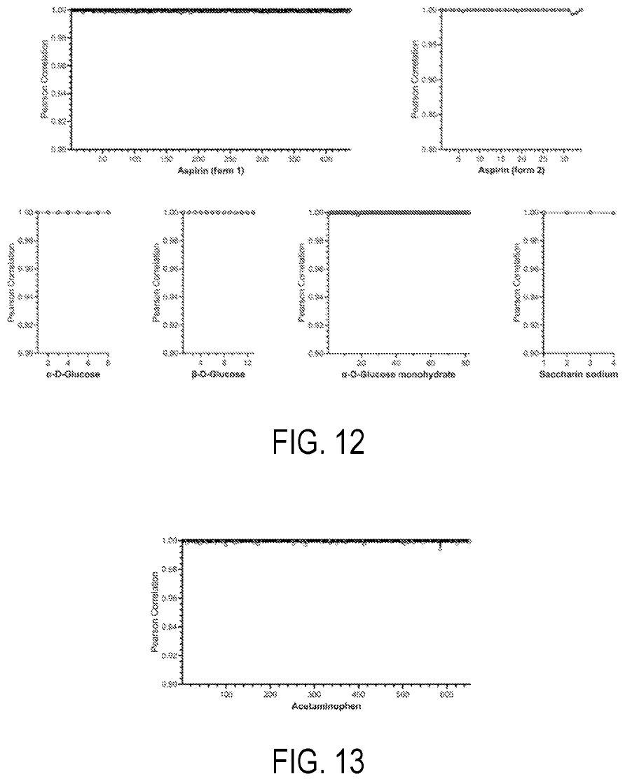

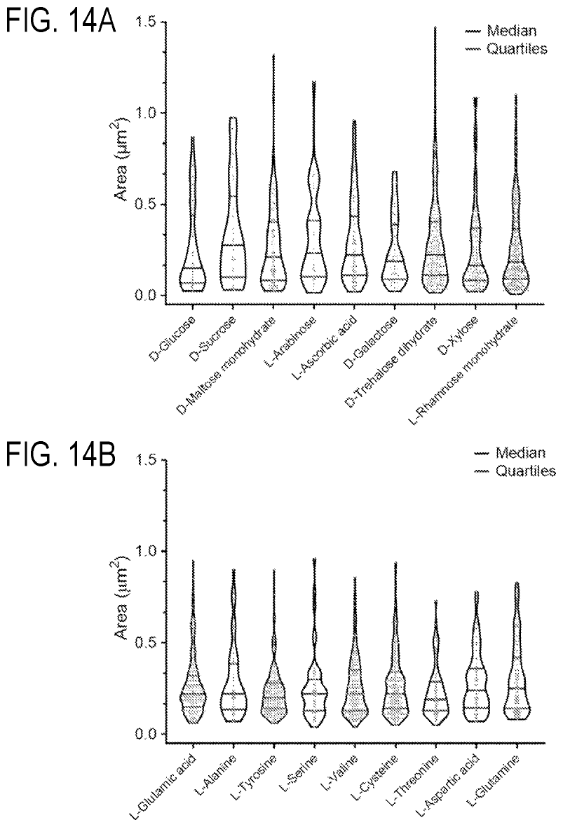

FIGS. 10-13 are Pearson correlation plots of unit cell parameters from processing in mixture B, mixture C, the aspirin tablet, and the acetaminophen tablet, respectively; and the reference cell parameters. FIGS. 14A-B are a series of crystal area distribution plots. In FIG. 14A, the crystal area distribution in mixture B is displayed. In FIG. 14B, the crystal area distribution in mixture C is displayed. Areas were converted from pixel² to μm² by the pixel sizes and magnification.

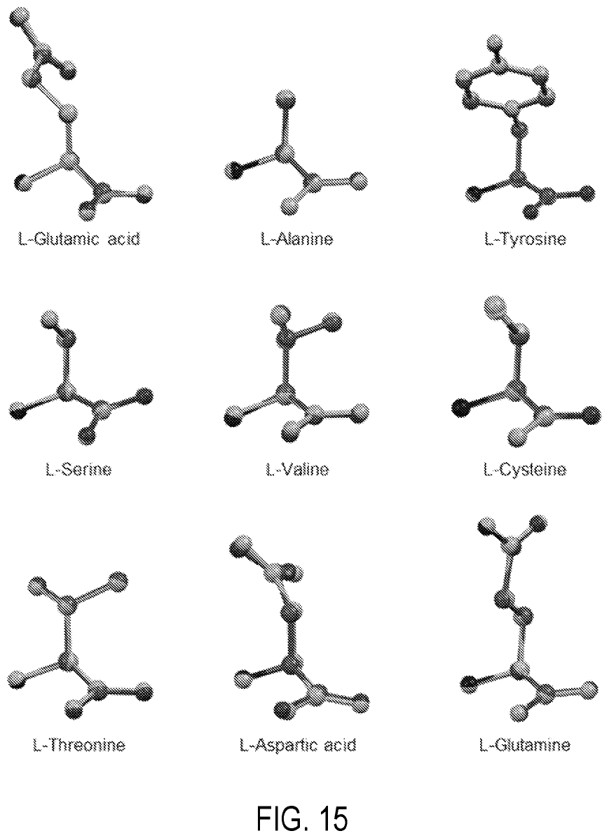

FIG. 15 is a series of crystal structures showing an overlay (Mercury) of the experimental solved structures in mixture C (colored by elements) and the literature-reported structures (colored in blue). The RMS values were as listed: L-Glutamic acid (0.058 Å), L-Alanine (0.016 Å), L-Tyrosine (0.033 Å), L-Serine (0.020 Å), L-Valine (0.062 Å), L-Cysteine (0.038 Å), L-Threonine (0.021 Å), L-Aspartic acid (0.105 Å), L-Glutamine (0.051 Å).

### Example Method

All the compounds were commercially purchased and used as received without further recrystallization. D-Glucose, D-Sucrose were purchased from Acros Organics. L-Valine was purchased from Alfa Aesar. Sodium bicarbonate, Magnesium sulfate heptahydrate, Sodium citrate dihydrate, Calcium acetate monohydrate, D-Galactose, L-Ascorbic acid were purchased from Fisher Chemical. Sodium sulfate, Potassium sulfate, Sodium chloride, Calcium gluconate, Magnesium acetate tetrahydrate, D-Maltose monohydrate, D-Trehalose dihydrate, L-Alanine, L-Arabinose, L-Aspartic acid, L-Cysteine, L-Glutamic acid, L-Glutamine, L-Rhamnose monohydrate, L-Serine, L-Threonine, L-Tyrosine were purchased from Sigma-Aldrich. D-Xylose was purchased from Tokyo Chemical Industry (TCI). The aspirin tablets (NDC: 59779-467-68) and acetaminophen tablets (NDC: 69842-484-62) were purchased from CVS pharmacy.

Compounds in Mixture A-C were carefully weighed by a Mettler Toledo (XPR225DR) analytical balance and mixed in a 20 mL scintillation vial. Mixture A was prepared as follows: Sodium bicarbonate (16.32 mg), Sodium sulfate (29.31 mg), Potassium sulfate (19.49 mg), Magnesium sulfate heptahydrate (9.14 mg), Sodium chloride (6.07 mg), Calcium gluconate (5.14 mg), Sodium citrate dihydrate (13.92 mg), Calcium acetate monohydrate (3.66 mg), Magnesium acetate tetrahydrate (8.93 mg). Mixture B was prepared as follows: D-Glucose (5.31 mg), D-Sucrose (3.88 mg), D-Maltose monohydrate (14.7 mg), L-Arabinose (5.42 mg), L-Ascorbic acid (17.55 mg), D-Galactose (7.3 mg), D-Trehalose dihydrate (28.05 mg), D-Xylose (14.73 mg), L-Rhamnose monohydrate (32.73 mg). Mixture C was prepared as follows: L-Glutamic acid (22.72 mg), L-Alanine (6.45 mg), L-Tyrosine (10.47 mg), L-Serine (6.62 mg), L-Valine (13.04 mg), L-Cysteine (24.95 mg), L-Threonine (5.5 mg), L-Aspartic acid (14.11 mg), L-Glutamine (14.13 mg) (Tables S3 and S4 in Supporting Information). The volume percent of each compound was calculated to generate a wide ratio range, from 3.0 to 27.5%. The compounds in the respective mixture were mixed together before being ground separately by an agate mortar and pestle set (internal diameter 50 mm) three times at room temperature to yield a fine powder without any visible crystalline solids left. The total weight of each mixture was more than 100 mg to ensure a thorough interaction with the agate mortar and pestle during the grinding process. One aspirin tablet (227.17 mg) and one acetaminophen tablet (566.26 mg) were weighed by Mettler Toledo (XPR225DR) analytical balance separately, and ground by an agate mortar and pestle set three times to yield the fine powders (same as mixture A-C).

The carbon-coated copper grids (400-mesh, 3.05 mm O.D., Ted Pella Inc.) were pretreated with glow-discharge plasma at 15 mA on the negative mode using PELCO easiGlow (Ted Pella Inc.), with no glow discharge for mixture A, 60 s glow discharge time for mixture B, aspirin tablet, and acetaminophen tablet, and 30 s for mixture C. Around 1 mg powder from each set was transferred to a 10 mL scintillation vial and separately mixed with the grid. After a gentle shaking of the vial, the grids were taken out and clipped at room temperature.

The clipped grids were loaded in an aligned Thermo-Fisher Talos Arctica Cryo-TEM (200 kV, ~0.0251 Å) at 100 K, equipped with a Falcon III direct electron detector (4096×4096 pixels). Intensity of 45.2% was found to be the condition for parallel beam during diffraction using the contrast of the objective aperture. For diffraction movies, the data was collected with an 829 mm diffraction length and using the smallest C2 aperture of 20 μm without the selected area aperture. The resulting beam size was approximately 1.5 μm. MicroED data was automatically collected using the SerialEM software in microprobe mode.

An atlas over the entire grid was collected as a low-magnification montage of 8×8 tiles. In the next step, typically more than 150 grid squares of interest were selected, and points were saved using “Add Points” in the SerialEM navigator window. After aligning the atlas with the magnification used in the medium magnification montages, one medium magnification montage of 3×3 pieces was collected at all saved points using the View mode. The “Rough eucentricity” and “Fine eucentricity” functions in SerialEM were used to automatically assign the eucentric height to each grid square that was stored with the corresponding maps. A point was added for each crystal of interest in the medium montage maps and saved in the navigator window. All points in the generated list were set up for data collection using the command “Acquire at items” function in the Navigator menu of SerialEM. TEM based techniques require samples thin enough to allow penetration of the electron beam and the purpose of selecting crystals in this size range only was to increase the chance of selecting crystals thin enough for an interpretable diffraction pattern. Care was taken during sample preparation to ensure a uniform grinding of the sample to achieve a homogeneous size distribution. Crystals ranging from 0.2 to 1.5 μm (size of the parallel beam) were picked for the data collection. Typical diffraction data using continuous rotation of the stage at 2°/s covering a total rotation range from −25° to +25° (−40° to +40° for aspirin and acetaminophen tablets) was automatically collected for each crystal using a SerialEM macro script. The script also used image mode to save an image of the content in the beam using the Search preset and was used to estimate crystal area and to visualize the crystal position during the MicroED data acquisition (see FIG. 1). During the rotation, the camera integrated frames continuously at a rate of ~0.5 s per frame, a total of 24.95 s exposure time for 50 frames (39.92 s for 80 frames).

An in-house developed Python script automatically processed the MicroED data via three steps using available software: (1) the raw MicroED data in MRC format were automatically converted to SMV format using mrc2smv software (available at https://cryoem.ucla.edu/downloads/snapshots); (2) the converted data were processed in XDS and (3) data sets were merged using XSCALE. XDS used a few settings in input: the detector distance is not refined together with the unit cell refinement due to the flatness of the Ewald's sphere, DELPHI was set to 30°, maximum errors was set to 10 and 3 for spot and spindle respectively, and occasionally MINIMUM_FRACTION_OF_INDEXED_SPOTS equals 0.1 was used to include weak data. The script evaluates different settings in XDS input for STRONG_PIXEL, SIGNAL_PIXEL, MINIMUM_NUMBER_OF_PIXELS_IN_A_SPOT, OFFSET, DATA_RANGE, SPOT_RANGE. The preferred merged data was found by evaluating all combinations of data sets to a certain maximal number of data sets included and the solutions were scored using statistics from XSCALE. The structures were solved using SHELXT and refined with SHELXL.

For the mixtures with the known crystal structure, the literature-reported unit cells in the Cambridge Structure Database (CSD) were used as a reference for the identification of a compound, as well as to phase data for the structure determination. The crystals were grouped according to compounds from the diffraction data and the relative ratios were calculated for each compound based on the number of dataset for each compound, which is referred to as the “counting ratio.” For the assignment of the proper crystal structure to an indexed data set, a maximum 1 Å length tolerance and 10° angle tolerance were used (see FIGS. 6-9 and Tables 13-17). These limits ensured that crystals were appropriately identified by the unit cells within reasonable error. The pattern of the unit cell parameters was unique for every crystal form and with no overlap between any two unit cells and could therefore be used to unambiguously assign the proper crystal structure to any indexed data set. For each sample a certain percentage of the data sets could not be assigned to a particular unit cell. This group contains images with no diffraction, smeared diffraction, multiple lattices or too low resolution for the software to be able to recognize the diffraction pattern. In case of no diffraction, the images recorded in parallel often showed that the beam had not hit the crystal properly.

The search images as saved in MRC format were converted to TIFF format using mrc2tif software. Then the converted images were imported into ImageJ software with the Threshold values to be adjusted until the whole crystals were fully detected (colored in red, FIG. 1). In ImageJ, the “Analyze Particles” function was used to automatically count the number of pixels corresponding to the crystal, (colored in black, FIG. 1) and area was manually inspected to ensure to only include the crystal area. The crystal area was calculated and summed for each component (S_{Comp}) and for each composition (S_{Total}) for ratio analysis.

The ratio of a compound in the mixture, referred to as the “area-corrected ratio” Ratio_{Obs} (see FIGS. 4A-D) was calculated by the following Equation (1):

$${Ratio}_{O b s} = \frac{{S}_{Comp}}{{S}_{Total}} \qquad ( 1 )$$

The ratio of the total area of one component (S_{comp}) to the total area of all crystals (S_{Total}) was determined for successfully indexed data sets only. The input ratio and observed ratio were plotted by Graphpad Prism 8.0.1 for Windows (GraphPad Software, San Diego, California USA, www.graphpad.com) as shown in FIGS. 4A-D.

### Discussion

Methods such as chromatography and mass spectrometry separate molecules based on difference in their sizes or chemical properties such as charge or hydrophobicity. For structures similar in size and chemical properties, like with isomers, separation is difficult. For crystalline samples the unit cell of a compound is a result of the exact packing of the molecules in order to lower the solid-state energy and is a stereoselective 3D process. Therefore, chemically similar compounds usually result in a very different crystal packing and sometimes also a different symmetry between the molecules, resulting in differences in space groups and unit cell parameters. As these can be determined rather accurately from processing the diffraction movies, the cells can easily be distinguished. Should the unit cell parameters accidentally be very close to each other for any two crystals, as a next step the difference between the individual intensities of two data sets can be used to discriminate between two structures, as understood from the fact that the intensity of a reflection hkl set can vary substantially with only a minor difference in the unit cell content. MicroED, just like the comparative example of PXRD, offers a way to analyze samples unrelated to chemical properties but rather from how they organize the molecules into a crystal. However, as noted above, MicroED provides advantages over the comparative method of PXRD.

One beneficial feature with MicroED is that there is no need to know the unit cells in advance. It allows for precise estimates of the unit cell parameters, either from the individual data sets or after merging of several data sets, as long as the rotation per crystal allows a correct indexing of the diffraction pattern. In contrast, the successful merging of a few data sets typically allows the determination of the MicroED structure so that a more complete analysis of the sample is achieved. Therefore, if the content of the unit cell as well as the dimensions of the unit cell is known, there is no need to prepare a standard with previously known peaks and the diffraction patterns and the structures needed can instead be determined on the fly. The ability to use single nanocrystals in MicroED and record the diffraction of a powder grain by grain resolves peak overlap due to several interfering diffraction patterns and brings diffraction on powder samples into a new level of accuracy. MicroED analysis of individual crystals can therefore be a powerful technique for complex samples with many constituents or otherwise overlapping powder diffraction patterns, yet with the same advantages as PXRD for identifying compounds in contrast to from their chemical profiles. This extends the use of diffraction based techniques for analysis of samples that are difficult to analyze from methods using hydrophobicity, charge or size as discriminating factors.

If the content of the unit cell is not known in advance and at the same time not being determined due to limited diffraction or other limitations, the size of the particles can be estimated and with a density of the samples the weight fraction can be calculated. In the studies described above, the visible area of each crystal was measured in order to estimate its volume, and final result is to some extent dependent on the size estimation of the diffracting crystals. However, even without a reliable way to estimate the diffracting volume properly as the thickness of the sample was not estimated, the results obtained from just counting the number of crystals is similar to the result where the area for each crystal is considered as well. As an example, the differences between the counted ratios and the area corrected ratios for each compound in sample B and C, is considerably smaller than the actual errors of the same ratios (o is 0.95% and 3.5% respectively). Therefore, it is possible to achieve close to the final result even without a careful volume estimation selecting crystals for analysis within a suitable size range in the selection process. As other errors, such as density calculation of the known ratios and substance handling errors, tend to be substantially less, it is believed that the largest error is related to the diffraction properties of the individual crystals and the crystallinity of the compounds. For PXRD analytical purposes, a 100% crystallinity can not be achieved and a detection limit of a few percent has been recorded for applications like PXRD, DSC and Raman.

The results of the analyses discussed above were compared with a round-robin assessment that was performed for PXRD in 2009. In that assessment Mannitol, Acetaminophen, and Silicon were used as a mixture containing two or three components at various amounts. While this comparative assessment mixture is much simpler in composition that the compounds tested using MicroED described above, with the PXRD tests the errors in compositional assessment were rather large (between 9-11%), while with MicroED, even though the samples were much more complex, the errors currently hover around 2-3%. This comparison suggests that MicroED should be considered as a complementary, or alternative, to PXRD.

The data collection and analysis in comparative examples can be time consuming for a large number of data sets. However, the automation of MicroED offers several advantages when analyzing mixtures of nano-sized crystals. In addition to the increased resolution and automatic structure determination, it is possible to retrieve the structures of the ingredients even for compounds in very small ratios, whereas in X-ray powder diffraction a structural analysis is substantially aided by an homogenous sample containing a single phase. Also, using MicroED there is no limit as to how many compounds that can be separated from a mixture, whereas overlap of the diffraction rings easily may limit the number of diffraction patterns that can be separated in X-ray powder diffraction.

In addition to crystallographic analyses, the efficacy of this approach may also depend on sample preparation. It was observed during sample preparation that the crystals had different physical properties, which influenced the efficiency of grid preparation. For instance, some materials were harder and less prone to breaking into the required size for MicroED, while others were brittle and more easily prepared. Secondly, electrostatic charging of the powder during grinding can cause an excess of powder to attach to the interior of the vial rather than onto the EM grid. Thirdly, the physical shearing of the sample may impact the crystallinity, although this is more often experienced with protein samples rather than small molecules or salts.

For a few compounds, behaviors were observed that are consistent with the compounds being hygroscopic. Some crystals of one compound appeared to be in a partially “melted” phase (see FIG. 1) and it was observed that some crystals clumped together more than otherwise seen, so they were not suitable for analysis. The carboxylic group of citric acid is slightly hygroscopic (can attract the equivalent of one water per molecule in equilibriumn), while D-Trehalose already contains crystalline water, whereas many sugar compounds are less hygroscopic. Analysis of hygroscopic nano crystals may be carried out in a humidity controlled environment.

For some crystals the diffraction was recorded but the diffraction was not enough for unit cell determination. These data points were considered as of undefined status and left out of the calculations as a whole. This diffraction could be mostly amorphous material that is partially more structured, in which case leaving them out of the calculation would be the consistent way to proceed. This situation is no different than any case where the crystalline portion is not close to 100%. It can also be the case that a few crystals were too small or there were several crystals with interfering diffraction, which then would underestimate the results for all compounds where this holds true. Target selection may be modified to account for such crystals to provide for diffraction analysis, avoiding the crystals that are visually problematic.

The experiments conducted using the three mixtures and two drug compositions have provided valuable insights into the factors that may impact compositional analysis. Notably, it was observed that around 70-80% of the selected crystals underwent automatic data processing and were successfully identified. However, the remaining crystals failed to produce results. Some crystals were too small to generate a high-quality diffraction pattern, making it difficult to determine the unit cell. Other crystals had a high margin of error in the estimation of the eucentric height, causing them to move out of the beam during continuous rotation MicroED. Furthermore, the carbon layer within a single grid square was not always flat, leading to fluctuations in the eucentric height. As a result, a few crystals partially rotated out of the beam center during data collection. Finally, some crystals exhibited smeared or streaky diffraction patterns or overlapped with other diffraction patterns, making it challenging to identify the correct unit cell. Therefore, some of the factors influencing the success of this approach include operator error in setting up the microscope. Improved crystal selection criteria and a local eucentric height determination may further improve the throughput.

In the above-described tests, a purpose was to analyze the constituents rather than to solve the structures, which is why a rotation range of 50° per crystal was used. This range facilitated the collection of more data sets within the same time frame, increasing the statistical significance of the analysis. For structure determination, a larger rotation range may be used. In general, continuous rotation of between 0° and 140°, and preferably at least 20°, per crystal allows unit cell determination without any a priori knowledge, which is suitable for analytic purposes. A rotation range of 50°-80° may provide for faster analysis. The standard deviations of the determined unit cell parameters are well within the tolerances (a maximum 1 Å deviation in length and 10° deviation in angles) used and the compounds with a correctly determined unit cell could be unambiguously assigned, as shown in FIGS. 6-9 and Tables 13-17. All determined unit cells showed a high degree of correlation to the reference values, as shown in FIGS. 10-13. These illustrations demonstrate that it is not necessary to solve the structures for compositional analysis, as proper unit cell parameters are sufficient to distinguish between the compounds in the mixtures. This approach also allows for a more comprehensive analysis of the sample when the structures of the constituents are already known, as indexing a data set is typically easier than solving the structure ab initio.

In structural biology it is important to collect multiple sets of redundant data, especially for systems with low signal-to-noise ratios. This has implications for MicroED. Firstly, for complex systems where only a fraction of a sample diffracts to the desired resolution, a significant part of the data collection process is dedicated to finding the best crystals. Secondly, redundant data can improve the signal-to-noise ratio and reduce the impact of systematic artifacts during data collection. Finally, merging datasets to increase completeness is often performed, but the outcome of this approach depends on the isomorphism of the data sets. In particular, because phasing using ab initio methods depends entirely on the structure factors, reliable estimates are required. A large pool of data sets facilitates the prospect of finding isomorphous data sets that could then be merged together to produce a complete data set for structure determination. This approach was demonstrated here with Mixture C where, despite the use of a short rotation range, all structures in mixture C were successfully solved as a step toward high throughput MicroED structure determination. The sample preparation, which comprises grinding the sample to a fine homogeneous powder, mixing the powder with an electron microscopy grid, and subsequently loading the grid in the microscope, is typically done in 15 min. The setting up of the low magnification atlas and the medium magnification montages is straightforward and requires another 15 min of human intervention and about 2-8 hours of automatic data collection in image mode, depending on the number of tiles of the medium magnification montage desired. The crystal selection in the above tests was done manually with another 2-4 man-hours which initiated the automatic data collection of about 750 crystals in, accounting for the remaining 15 hours of a 24-hour shift of the microscope. The script, which automatically processes each of the data sets and merges combinations of the data sets collected, is finished within 12-72 hours depending on the settings and the hardware used. The result of the composition analysis and the processing is summarized in a few text files. Thus, the whole process typically takes 2 days on 750 data sets and requires a few hours of manual work mostly for the crystal selection process.

The present disclosure presents an automatic approach to MicroED using available software. The data collection process requires no human intervention after initial setup, making it suitable to run during less busy microscope shifts. This approach is built on the CryoEM data collection software, SerialEM. By using available software, more laboratories may be able to implement a higher level of automation in their MicroED data collection processes. In summary, the high throughput automatic MicroED approach established and demonstrated in this disclosure provides for expanding the applications of MicroED as an analytical tool well beyond a structural determination tool. With the ability to collect and analyze vast amounts of data, MicroED can be used for compositional analysis, providing a reliable and statistically significant analysis of the relative composition of a sample.

### Example System

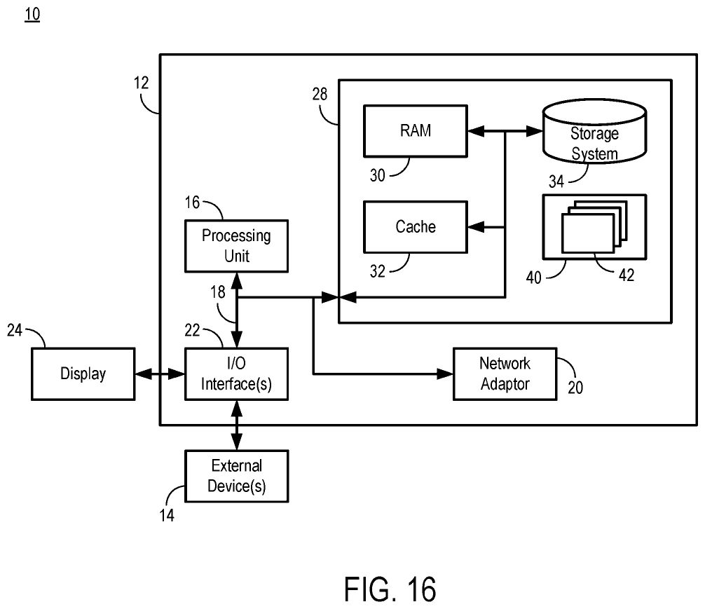

Referring now to FIG. 16, a schematic of an example of MicroED system, implemented as a computing node, is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 16, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16. Computer system/server 12 may include a variety of computer system readable media. Such media may be any available media that is accessible by computer system/server 12, and it includes both volatile and non-volatile media, removable and non-removable media. Computer system/server 12 is an example of a controller in accordance with the present disclosure.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of examples of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of examples as described herein.

External devices 14 may include MicroED external devices such as an electron microscope configured to receive a grid (e.g., the grid described above). Computer system/server 12 may also communicate with one or more user interface external devices such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The computer program product provided herein may include a computer readable storage medium (or media) having computer readable program instructions thereon for causing a processor to carry out aspects of the present disclosure. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an EPROM, a Flash memory, a SRAM, a portable CD-ROM, a DVD, a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. In one example, the programming language is Python. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet using an Internet Service Provider). In some examples, electronic circuitry including, for example, programmable logic circuitry, FPGAs, or PLAs may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

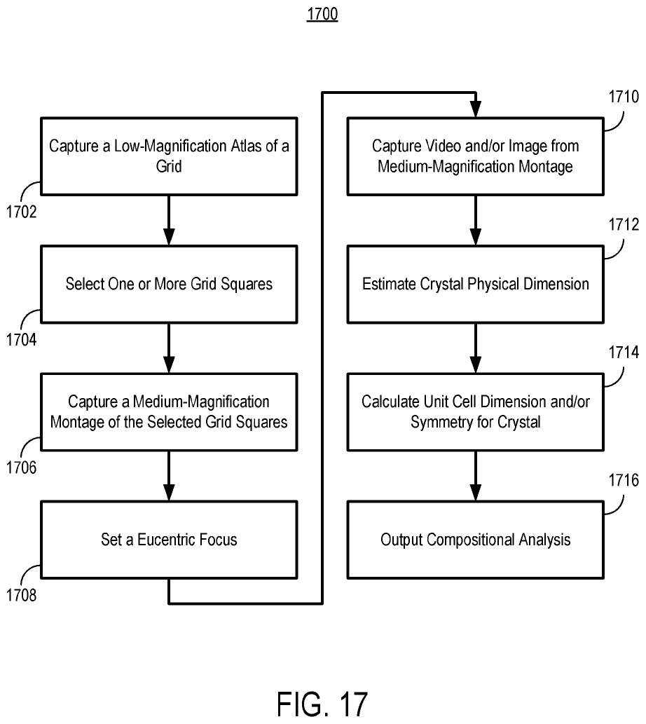

FIG. 17 illustrates an example process 1700 for autonomous compositional analysis via a MicroED system. Process 1700 is one example of the flow illustrated in FIG. 1. In some implementations, process 1700 may be automatically performed by system 10 illustrated in FIG. 16; however, in other implementations process 1700 may be performed by any system capable of collecting electron microscope images of a grid and having a controller and/or memory that enable the system to automatically perform the operations of FIG. 17. The controller and/or memory may be integral with the device capable of collecting electron microscope images, may be external thereto (e.g., cloud-based), or combinations thereof. The automation of the operations of process 1700 allow for high throughput and the use of large volumes of data to perform analytical investigations on complex mixtures, in contrast to manual or semi-automated comparative examples which are feasible only on comparatively simple mixtures.

Process 1700 includes operation 1702 of capturing a low-magnification atlas of a grid. At operation 1704, process 1700 includes selecting one or more grid squares from the low-magnification atlas. Operation 1704 may include counting distinct crystals within a grid square of the grid, adding the grid square to a list where it is determined that the distinct crystals are suitable for analysis, and repeating these operations until a predetermined number (e.g., 20 or more) of distinct crystals is reached. The determination as to whether the distinct crystals are suitable may be made based on any combination of size, appearance and opacity under magnification of the distinct crystals. For example, transmitted light levels (e.g., gray levels) may be measured and used to estimate thickness. Operation 1706 includes capturing a medium-magnification montage of the grid based on the selected one or more grid squares. In general, “low-magnification” may be any magnification that is lower than the medium-magnification montage, and “medium-magnification” may be any magnification that is higher than the low-magnification atlas. In one particular example, “low-magnification” may be 10×.

Operation 1708 includes setting a eucentric focus of the electron microscope. Operation 1710 includes capturing video and/or images (e.g., a continuous rotation MicroED video) of at least one crystal from the medium-magnification montage. For example, the video may include a plurality of images collected over a predetermined continuous rotation range (e.g., 0°-140°, such as) 50°-80°, wherein respective ones of the plurality of images are captured at predetermined rotation intervals (e.g., 2° or rotation between successive image captures). At operation 1712, process 1700 includes estimating a crystal physical dimension of the at least one crystal base don the captured video and/or images. The estimate may be based on a remeasuring of the selected one or more grid squares. The estimated crystal physical dimension may be used at operation 1714 to calculate at least one of a unit cell dimension and a symmetry for the at least one characteristic. The calculated quantity may be referenced using a database to correlate the composition with a new compound. For example, a diffraction pattern may be received (e.g., from the video and/or images of operation 1710) and matched to a corresponding crystal composition in the database.

At operation 1716, a compositional analysis is output. The compositional analysis may include at least one of a breakdown of the mixture by structure, percentage, molar ratio, or weight; an indication of one or more structures of components of the mixture; a comparison of the compositional analysis with a reference (input) compositional analysis; a graph indicating a number of crystals identified for each compound of the mixture; a crystal area dimension plot for each compound of the mixture; and combinations thereof.

Although the invention has been described and illustrated in the foregoing illustrative aspects, it is understood that the present disclosure has been made only by way of example, and that numerous changes in the details of implementation of the invention can be made without departing from the spirit and scope of the invention, which is limited only by any allowed claims that are entitled to priority to the subject matter disclosed herein. Features of the disclosed aspects can be combined and rearranged in various ways.

Other examples and uses of the disclosed technology will be apparent to those having ordinary skill in the art upon consideration of the specification and practice of the invention disclosed herein. The specification and examples given should be considered exemplary only, and it is contemplated that the appended claims will cover any other such aspects or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its aspects.

1-54. (canceled)

55. A microcrystal electron diffraction (MicroED) microscopy system, comprising:
an electron microscope configured to receive a grid, the grid including a mixture of compounds; and
a controller configured to automatically:
cause the electron microscope to capture a low-magnification atlas of the grid,
select one or more grid squares from the low-magnification atlas,
cause the electron microscope to capture a medium-magnification montage of the grid based on the selected one or more grid squares,
set a eucentric focus of the electron microscope,
cause the electron microscope to capture a continuous rotation MicroED video of at least one crystal from the medium-magnification montage,
estimate a crystal physical dimension of the at least one crystal, based on the continuous rotation MicroED video,
calculate at least one of a unit cell dimension and a symmetry for the at least one crystal, based on the estimated crystal physical dimension, and
output a compositional analysis of the mixture of compound based on the at least one of the unit cell dimension and the symmetry.

56. The system of claim 55, wherein the controller is further configured to automatically search for a crystal unit cell dimension in a database, based on the calculated at least one of the unit cell dimension and the symmetry.

57. The system of claim 55, wherein the controller is configured to automatically select one or more grid squares from the low-magnification atlas by automatically:
counting distinct crystals within a grid square of the grid;
in response to a determination that the distinct crystals are suitable for analysis, adding the grid square to a list; and
repeating the operations of counting and adding until a predetermined number of distinct crystals is reached.

58. The system of claim 57, wherein the determination that the distinct crystals are suitable for analysis is based on at least one of a size of the distinct crystals, an appearance of the distinct crystals, or an opacity of the distinct crystals under magnification as determined by a measurement of transmitted light levels.

59. The system of claim 55, wherein the continuous rotation MicroED video includes a plurality of images collected over a total predetermined continuous rotation range, wherein respective ones of the plurality of images are captured at predetermined rotation intervals, and correspond to frames of the MicroED video.

60. The system of claim 55, wherein the controller is configured to automatically estimate the crystal physical dimension by remeasuring the selected one or more selected grid squares.

61. The system of claim 55, wherein the controller is further configured to automatically receive a diffraction pattern and to match the diffraction pattern to a corresponding crystal composition in a database.

62. A microcrystal electron diffraction (MicroED) microscopy method, comprising automatically:
capturing, by an electron microscope, a low-magnification atlas of a grid, the grid including a mixture of compounds;
selecting one or more grid squares from the low-magnification atlas;
capturing, by the electron microscope, a medium-magnification montage of the grid based on the selected one or more grid squares;
setting a eucentric focus of the electron microscope;
capturing, by the electron microscope, a continuous rotation MicroED video of at least one crystal from the medium-magnification montage;
estimating a crystal physical dimension of the at least one crystal, based on the continuous rotation MicroED video;
calculating at least one of a unit cell dimension and a symmetry for the at least one crystal, based on the estimated crystal physical dimension; and
outputting a compositional analysis of the mixture of compound based on the at least one of the unit cell dimension and the symmetry.

63. The method of claim 62, further comprising automatically searching for a crystal unit cell dimension in a database, based on the calculated at least one of the unit cell dimension and the symmetry.

64. The method of claim 62, wherein the operation of automatically selecting one or more grid squares from the low-magnification atlas includes automatically:
counting distinct crystals within a grid square of the grid;
in response to a determination that the distinct crystals are suitable for analysis, adding the grid square to a list; and
repeating the operations of counting and adding until a predetermined number of distinct crystals is reached.

65. The method of claim 64, wherein the determination that the distinct crystals are suitable for analysis is based on at least one of a size of the distinct crystals, an appearance of the distinct crystals, or an opacity of the distinct crystals under magnification as determined by a measurement of transmitted light levels.

66. The method of claim 62, wherein automatically capturing the continuous rotation MicroED video includes automatically capturing a plurality of images collected over a total predetermined continuous rotation range, wherein respective ones of the plurality of images are captured at predetermined rotation intervals, and correspond to frames of the MicroED video.

67. The method of claim 66, wherein the predetermined rotation intervals are greater than or equal to 0.01° and less than or equal to 5° per frame of the MicroED video.

68. The method of claim 67, wherein the total predetermined continuous rotation range is greater than or equal to 0° and less than or equal to 140°.

69. The method of claim 62, wherein the operation of automatically outputting the compositional analysis includes at least one of: outputting a breakdown of the mixture by structure, percentage, molar ratio, or weight; outputting one or more structures of components of the mixture of compound; outputting a comparison of the compositional analysis with a reference compositional analysis; outputting a graph indicating a number of crystals identified for each compound of the mixture of compounds; or outputting a crystal area dimension plot for each compound of the mixture of compounds.

70. A non-transitory computer-readable medium storing instructions that, when executed by at least one processor of a microcrystal electron diffraction (MicroED) system, cause the system to automatically perform operations comprising:
capturing, by an electron microscope of the MicroED system, a low-magnification atlas of a grid, the grid including a mixture of compounds;
selecting one or more grid squares from the low-magnification atlas;
capturing, by the electron microscope, a medium-magnification montage of the grid based on the selected one or more grid squares;
setting a eucentric focus of the electron microscope;
capturing, by the electron microscope, a continuous rotation MicroED video of at least one crystal from the medium-magnification montage;
estimating a crystal physical dimension of the at least one crystal, based on the continuous rotation MicroED video;
calculating at least one of a unit cell dimension and a symmetry for the at least one crystal, based on the estimated crystal physical dimension; and
outputting a compositional analysis of the mixture of compound based on the at least one of the unit cell dimension and the symmetry.

71. The non-transitory computer-readable medium of claim 70, the operations further comprising searching for a crystal unit cell dimension in a database, based on the calculated at least one of the unit cell dimension and the symmetry.

72. The non-transitory computer-readable medium of claim 70, wherein the operation of selecting one or more grid squares from the low-magnification atlas includes:
counting distinct crystals within a grid square of the grid;
in response to a determination that the distinct crystals are suitable for analysis, adding the grid square to a list; and
repeating the operations of counting and adding until a predetermined number of distinct crystals is reached.

73. The non-transitory computer-readable medium of 70, wherein the predetermined number is greater than or equal to approximately 20.

74. The non-transitory computer-readable medium of claim 70, the operations further comprising receiving a diffraction pattern determined from the continuous rotation MicroED video and matching the diffraction pattern to a corresponding crystal composition in a database.
